(19) United States
(12) Patent Application Publication
(10) Pub. No.: US 2025/0041478 A1
(43) Pub. Date: Feb. 6, 2025

(54) FIBROUS POLYPEPTIDE-BASED BIOSCAFFOLD DELIVERY OF THERAPEUTICS TO NEURAL CELLS

(71) Applicant: The Regents of the University of Colorado, A Body Corporate, Denver, CO (US)

(72) Inventors: An-Jey Su, Denver, CO (US); Kia Washington, Denver, CO (US); Wei-Fang Su, Denver, CO (US); Yu-Ching Huang, New Taipei City (TW)

(21) Appl. No.: 18/734,972

(22) Filed: Jun. 5, 2024

Related U.S. Application Data
(60) Provisional application No. 63/506,336, filed on Jun. 5, 2023.

(51) Int. Cl. A61L 27/18; A61L 27/54

(52) U.S. Cl. CPC A61L 27/18; A61L 27/54; A61L 2300/252; A61L 2300/406; A61L 2300/414; A61L 2430/32

(57) ABSTRACT

Disclosed herein are compositions and methods for aiding in the treatment of various reversible and irreversible neurodegenerative diseases. In some embodiments, poly(γ-benzyl-L-glutamate)(PBG) and/or its hydrolyzed copolymer (poly(γ-benzylglutamate) 80-r-(γ-glutamic acid)20)(PBGA) may be used to make the disclosed fibrous scaffolds, for one example by use of an electrospinning process. The disclosed materials may be biocompatible molecules, for example polypeptides. In some embodiments the disclosed materials may contain a neurotransmitter, in one embodiment glutamate. In some embodiments, the disclosed scaffolds may consist of one or more aligned fibers. In some embodiments, the scaffold may include one or more active compounds. In some embodiments, the active compound may be neuroprotective, for example a neuroprotective antibiotic. In some embodiments, the active compound may be minocycline hydrochloride (MH). In many embodiments, the disclosed scaffold may aid in stabilizing the active compound.

### CROSS-REFERENCE TO RELATED APPLICATIONS

This application claims priority to U.S. Provisional Patent Application No. 63/506,336 filed Jun. 5, 2023, entitled “FIBROUS POLYPEPTIDE-BASED BIOSCAFFOLD DELIVERY OF THERAPEUTICS TO NEURAL CELLS” which is incorporated by reference herein, in the entirety and for all purposes.

### BACKGROUND

Tissue engineering (TE) is a strategy employed to improve the outcomes of surgical repair, reconstruction or transplantation to restore loss of function in pathological tissue or whole organs subjected to traumatic injury or disease. In TE, biocompatible materials are used in fabricated devices which interact with cells to potentiate their intrinsic regenerative capacity to repair damaged tissue. Cell survival, and differentiation are essential to regenerative responses to repair tissues. Scaffolds are engineered with architecture to support these processes.

TE scaffolds can be applied to any tissues that require restoration, and are typically composed of natural or synthetic biopolymers each showing positive benefits in different applications. They can be functionalized with drugs, proteins or peptides to provide cellular environment that enhances the restoration of tissues and organs. Polymeric hybrids of natural and synthetic molecules as the scaffold backbone has emerged as a TE strategy that might allow more ubiquitous application across various physiological systems such and tissue types. Among the major body systems, injuries to the mammalian nervous system remains the least responsive to treatment and has to poorest prognosis when severely pathological.

Pathologies of the nervous system are numerous, arising from various etiologies including inherited, chronic neurodegenerative diseases such as Alzheimer's disease, Parkinson's disease, and Huntington's disease. Such disorders are considered incurable, and largely untreatable diseases of the central nervous system (CNS) due to progressive neuronal cell loss in specific brain regions. Recovery from neurodegenerative diseases and acute injuries to the mammalian CNS is complicated by the inability of lesioned axons to regenerate. Outcomes for patients suffering from peripheral nerve injury (PNI) also remain sub-optimal, regardless of surgical intervention, due to distal axonal degeneration following PNI. The use of biomaterials to address the challenges associated with axonal repair in both CNS and peripheral nervous system (PNS) have been actively investigated for decades. Despite the promise of these neural tissue engineering strategies, the need for new highly effective clinical therapies remains unmet.

### SUMMARY

Disclosed herein are methods and systems for tissue engineering and repair, for example of neuronal cells and tissue. In many embodiments, the disclosed compositions, devices, and systems may be useful in delivering one or more active compounds to developing tissue and/or cells.

Also disclosed are various systems for supporting mammalian cells, comprising: a scaffold comprising a plurality of fibers, wherein the majority of fibers are oriented substantially parallel; and at least one active compound, for example neuronal growth factor. In some embodiments, the system may comprise a neuronal or kidney cell, and/or the fibers may comprise one or more of poly(γ-benzyl-L-glutamate)(PBG), hydrolyzed copolymer (poly(γ-benzylglutamate) 80-r-(γ-glutamic acid)20)(PBGA), and minocycline hydrochloride (MH).

Also disclosed are various compositions comprising: a polymer comprising at least one neurotrophic factor, and at least one active compound, and the polymer may be selected from poly(γ-benzyl-L-glutamate)(PBG), and hydrolyzed copolymer (poly(γ-benzylglutamate) 80-r-(γ-glutamic acid)20)(PBGA); and the at least one active compound is minocycline hydrochloride (MH). In some embodiments, the composition may be fabricated as a fiber.

Also disclosed are methods, for example methods of forming a scaffold for growth or support of a mammalian cell, comprising: combining a polymer selected from poly(γ-benzyl-L-glutamate)(PBG), and hydrolyzed copolymer (poly(γ-benzylglutamate) 80-r-(γ-glutamic acid)20)(PBGA), with at least one active compound, which, in some embodiments may be minocycline hydrochloride (MH); forming a fiber from the polymer composition; and arranging the fiber to create a scaffold. In some embodiments, the forming and arranging may be accomplished using electro spinning and/or the concentration of the active compound is less than about 5% by weight. In some embodiments, the method may be a method of treating a peripheral nerve injury, comprising: identifying a mammalian subject having an injury to a nerve or nerve tissue; surrounding the injured nerve or nerve tissue with the disclosed systems or the compositions. In some embodiments, the injury is to a sciatic nerve.

Also disclosed are various devices comprising: fiber scaffold comprising; a polymer; and an active compound. In some embodiments, the polymer may be selected from poly(γ-benzyl-L-glutamate)(PBG), and poly(γ-benzylglutamate) 80-r-(γ-glutamic acid) 20 (PBGA) and/or the active compound may be minocycline hydrochloride (MH). In some embodiments, the active compound is less than about 5% by weight of the fiber and/or release of the active compound from the fiber may not show two-phase kinetics after 24 hours, and/or at least about 50% of the fibers may be oriented in the same or the opposite direction. In some embodiments, the disclosed device may be used in treating a peripheral or central nervous system cell or tissue.

### BRIEF DESCRIPTION OF THE DRAWINGS

FIG. 1 is a schematic diagram of one embodiment of the disclosed drug delivery system, comprising electrospun fibrous polymer scaffold and minocycline hydrochloride (MH) for neural tissue engineering.

FIGS. 2A-2B depicts one embodiment of producing the disclosed (FIG. 2A) PBG and (FIG. 2B) PBGA polymers.

FIGS. 3A-3D are photographs of (FIG. 3A) PBGA (FIG. 3B) PBGAMH2 (FIG. 3C) PBGAMH4 (FIG. 3D) PBGAMH6 scaffold. The addition of MH can be directly observed by the appearance of yellow color. The yellow becomes intense as the concentration of MH increases.

FIGS. 4A-F shows Minocycline hydrochloride release profiles from PCL, PBG, and PBGA scaffolds with three different concentrations in 7 days and the kinetic model fitting results. Release profiles of % cumulative release versus time are shown in (FIG. 4A)(FIG. 4B)(FIG. 4C). Kinetic model fittings are done by plotting nature log value of cumulative release (In) versus of nature log time (Int) as shown in (FIG. 4D)(FIG. 4E)(FIG. 4F). Release time points are measured at 2, 4, 8, 24, 48, 96, and 168 hours. n=3 for each group.

FIG. 5 shows biocompatibility of PC12 cells on polymer scaffold after 5-day culture in terms of mean average ratio of live cells to dead cells (left) and cell viability index (right). PBG series scaffolds show higher live/dead ratio than control group (coverslip) and PCL series scaffolds, indicating better biocompatibility. Each group was repeated three times. 5-6 pictures were taken at different spot for each group, then the average value was used to make the plot as shown in FIG. 3A. The cell viability was determined by normalizing the percentage of cell reduction on each scaffold to the coverslip as control at day 1. Each group was repeated for 3 times. H test: *** indicates p≤0.001, ** indicates p≤0.01, and * indicates p≤0.05.

FIG. 6 shows SEM images of PC12 cell growth and cell differentiation (with 100 ng/mL NGF) for five days on PBG, PBGA, PBGMH4, and PBGAMH4 scaffold. PC12 cells demonstrate good adhesion on the scaffolds. With the addition of NGF, the neurite outgrowth extends along the direction of the fibers. Scale bar=10 μm.

FIG. 7 is a box chart of PC12 neurite lengths after 5-day differentiation. Each group was repeated for 3 times. 10-20 fluorescence pictures were taken from different area of each group. Neurite counts=77-126 in each group. H test: *** indicates p≤0.001, ** indicates p≤0.01, and * indicates p≤0.05.

FIG. 8 are graphs of the average median neurite length and longest neurite length of PC12 cells after 5-day differentiation. Each group was repeated for 3 times. 10-20 fluorescence pictures were taken from different area of each group. Neurite counts=77-126 in each group. The only one neurite length ranking in the middle was selected as the median neurite length (left figure). The longest 10% of neurites of each group were selected and averaged as the longest neurite length (right figure).

FIG. 9 are graphs showing top side fiber diameter variation in PBG and PBGMH4 scaffold felts over a storage period of 12 months.

FIG. 10 is an SEM top view images of PBG and PBGMH4 fiber scaffolds over a storage period of 12 months

FIGS. 11A-11B is an SEM cross-section images of PBG scaffolds felts (FIG. 11) just completed by electrospinning and (FIG. 11B) after 9 months of storage.

FIGS. 12A-12D are SEM images of the (FIG. 12A) top side and (FIG. 12B) bottom side of PBG scaffold felts, as well as the (FIG. 12C) top side and (FIG. 12D) bottom side of PBGMH4 scaffold felts

FIG. 13 are graphs of maximum tensile strength variation of PBG and PBGMH4 scaffold over a storage period of 12 months.

FIG. 14 are graphs showing cumulative drug release profiles of PBGMH4 scaffolds with various storage times (left) and cumulative drug release profiles of PBGMH4 fresh electrospun scaffolds and storage 7 months after tensile strength test (right).

FIG. 15 shows a calibration curve of MH in PBS solution.

FIG. 16 is a bar graph of cell viability.

FIG. 17 is a study design and tested groups.

FIGS. 18A-18C shows graphed results from studies in Example 4.

FIG. 19A is a table of results and FIG. 19B is a graph.

FIG. 20A is a table of muscle weight and 20B are photos showing same.

FIG. 21 is a table of treatment groups.

FIG. 22 is a table comparing PBGMH4 and PBGAMH4 over 6 weeks.

### DETAILED DESCRIPTION

Disclosed herein are methods, systems, devices, and compositions for treating nerve cell or nerve tissue damage resulting from various injuries, diseases, and conditions.

Neural tissue engineering combines strategies from cell biology, material science, and engineering to fabricate scaffolds for clinical use. Scaffolds are engineered to mimic the structure and function of natural tissues to facilitate cell repair by delivery of bioactive molecules or drugs that accelerate regeneration of the injured or dead tissue. The regenerative capability of artificial scaffolds employed in neural engineered tissue strategies is predicated on biocompatibility, biodegradability, mechanical structure, and extracellular matrix structure mimicry. Electrospinning is a versatile technique to fabricate polymer solutions under high voltage into micro or nanofibers that comprise fibrous scaffolds. Such prepared scaffolds are commonly used in tissue engineering and drug delivery. The desired fiber diameter of scaffolds can be adjusted by changing parameters such as applied voltage, polymer solution concentration, solvent ratio, and flow rate of the solution. The advantages of fabricating scaffolds by electrospinning technique include easy operation and the adaptability of the process. The simple electrospinning process can produce fibrous scaffolds from lab-scale to industrial-scale with specific fiber orientation and composition, making it practical for medical application.

Mechanisms of neuronal cell death are shared across various neurodegenerative diseases or trauma, with a cascade of cell death signaling involving oxidative stress, mitochondrial dysfunction and caspase activation, ultimately resulting in apoptosis and other processes such as necroptosis. Neuroprotective strategies often seek to slow neuron loss associated with neurodegenerative diseases, strokes, traumatic brain injury, spinal cord injury, etc. Minocycline hydrochloride (MH) is a second-generation semi-synthetic tetracycline, with antibiotic properties that inhibit protein synthesis in susceptible organisms. Beyond its antibiotic properties, MH has anti-inflammatory and anti-apoptotic properties. The neuroprotective effect of MH has been reported in both experimental models and clinical trials. Additionally, MH is reported to potentiate nerve growth factor (NGF)-induced neurite outgrowth in PC12 cells depending on concentration. In experimental animal models, dosages for MH range from 10-50 mg/kg initial dosage (i.v., intravenously; i.p., interperitoneally; v.o. voluntarily oral) with supplemental doses at 12-24 h (q12h-24h). Clinically, MH is administered for bacterial infections at 200 mg/day, followed by 100 mg/12h (i.v., v.o., q12h). For acute spinal cord injury, 400 mg is given intravenously (i.v., q12h) with an 800-mg loading dose. In either case, repeated dosing is necessitated by the relatively short half-life of MH in vivo (plasma half-life ranges from 11-23 hours). Therefore, controlled and sustained drug-delivery to local targets is desirable for administration of MH. Material scientists can fabricate and modify synthetic polymers as fibrous scaffolds with specific biological and mechanical strength properties to customize their application for tissue engineering. Scaffolds can be loaded with bioactive molecules, including immunosuppressive drugs, growth factors or anti-inflammatory molecules for delivery to specific tissues. An optimized drug delivery system from fibrous scaffolds should have homogeneous distribution of a drug (or other bioactive molecule) amongst the fibers, predictable drug release at a determined rate, and drug stability when incorporated into the system over a range of temperatures and predictable period. Thus, the compatibility between drug and polymer is a critical consideration in designing these systems.

Applicants have demonstrated that poly(γ-benzyl-1-glutamate)(PBG) and (poly(γ-benzylglutamate) 80-r-(γ-glutamic acid)20)(PBGA) biomaterials can successfully promote neurite outgrowth. Here, Applicants show fabrication and use of aligned PBG and PBGA polypeptide scaffolds containing the neuroprotective drug MH. The release profiles of MH from the disclosed scaffolds were systematically studied. Applicants show that the release profile is related to the interfacial behaviors between drug and polypeptide based scaffold materials. Applicants hypothesized that adding MH to the scaffolds would enhance NGF-induced PC12 neurite outgrowth. Further, Applicants predicted that the extent of enhancement would depend on anisotropy and the MH concentration in the scaffold. The results disclosed below show that a tissue engineering scaffold-drug delivery system based on polypeptide-fibrous scaffolds containing neuroprotective MH are useful in supporting neural regeneration and improving outcomes in previously untreatable diseases and conditions.

Disclosed herein is a drug delivery system comprising a scaffold device composed of a composition of polypeptide-based electrospun fibers and the neuroprotective antibiotic MH. The disclosed systems, devices, and compositions may be useful in treating various diseases and conditions, as well as for neural tissue engineering.

In many embodiments, the disclosed scaffolds may be comprised of two polypeptides PBG or PBGA, which contain neuronal stimulant glutamate and glutamic acid. In many embodiments, use of PBG and/or PBGA may result in enhanced release kinetics of one or more bioactive compounds compared to other natural (collagen, decellularazed ECM) or synthetic polymeric scaffolds, for example scaffolds of poly-capro lactone PCL, PGLA or PEUU.

The release profiles of the neuroprotective antibiotic MH from the disclosed scaffolds were shown to be surprisingly better than existing options. Applicants hypothesized that this may be due to strong hydrogen bonding of PBGA scaffold and hydrophobicity of PBG scaffold. These characteristics resulted in MH showing surprisingly sustained release from these novel scaffolds, especially when compared with release from PCL scaffold. The release profiles for the disclosed scaffolds were shown to follow Fick's diffusion law.

Applicants show, herein that the disclosed systems, devices, compositions, and methods are effective at enhancing neuronal cell viability and differentiation. Specifically, the ability of the scaffolds to enhance cell viability and differentiation on were investigated using neuronal PC12 in in vitro experiments. Applicants compared scaffolds comprised of fibers fabricated from different materials, and with various MH concentrations. Specifically, of the combinations tested, PBGA scaffold with 4 wt. % MH demonstrated the highest cell viability, suggesting that neurotransmitter-like functional group (glutamic acid) and adequate MH concentration enhances biocompatibility. However, Applicants showed that cell viability and neurite length decreased at high MH concentrations, for example as MH concentration increased to 6 wt. % cell viability was negatively affected. Results in animal models or clinical applications may differ. After five days of cell differentiation, the measured neurite outgrowth also appeared highest with PBGA scaffold and mid-range MH concentrations. In these studies, neurite length median of this scaffold reaches 62.71 μm, and the maximum length was 190.29 μm. This represents a four-fold increase over median neurite length produced on PBGA only scaffold (47.07 μm). Altogether, the results indicate that the PBGA scaffold with 4 wt. % of MH (PBGAMH4) represents a promising drug delivery system without external stimulations in cell culture for neural tissue engineering. This novel biocompatible and bio-functional scaffold is not complex (in terms of chemical composition and synthesis) and has never been reported in the literature. The disclosed iteration of the novel scaffolds are fabricated from polypeptides and engineered to provide a built in drug delivery of MH and nerve cell stimulating moiety of glutamate. They have potential applications in various neural tissue engineering methods, for example methods to repair and/or regenerate cells and tissue of the central nervous system or peripheral nervous system.

The disclosed systems, devices, compositions, and methods comprising PBGMH & PBGAMH are stable for long periods of time. In many embodiments, the disclosed scaffolds and compositions may be stable for long periods of time without refrigeration. This enhanced stability affords the scaffolds and fibers enhanced utility as nerve bioconduits for delivery of various compounds and factors, for example regenerative molecules to promote directional neural tissue repair. Stability at room temperature provides enhanced delivery of the scaffold utility in austere environments or environments that lack typical infrastructure found in clinics serving densely populated areas. Existing bioconduits, for example those made of elastomeric materials such as PCL and PEUU, do not include the present scaffolds stimulatory elements such as glutamate and furthermore, existing materials are not ordered and cannot provide directional guidance for regenerating neurites.

PBG and PBG-MH were evaluated as nerve-wraps in a sciatic nerve injury (SNI) model. PBG and PBG-MH were shown to support repair of about 10 mm nerve gaps with PBG-MH outperforming PBG in accelerated functional recovery as measured by SFI. EMG and final muscle weight showed improved neuro-regeneration in both wrap groups over 12-weeks recovery. An acceleration of neuro-regeneration was observed in the treatment group vs the no treatment group at four weeks post-operatively in both behavioral testing and electrophysiological testing. However, by six weeks the two groups had leveled out in the extent of recovered neuronal function. Additionally, the gastrocnemius muscle appeared to retain more mass at the conclusion of the study. Taken together, the results suggest that PBG-MH or possibly PBGA-MH to be an excellent candidate for neural guide conduits (NGC) modalities to treat PNI.

Long term monitoring and analysis of the storage stability of PBG and PBGMH4 fiber scaffold was untertaken, Applicants show that PBGMH4 exhibits superior stability during storage. This enhanced stability is attributed to the presence of MH in PBGMH4, which may induce electrostatic repulsion between fibers and prevent merging of fibers. In contrast, PBG-only fiber scaffold felts showed a significant increase in fiber diameter with extended shelf storage, leading to collapse of the microstructure. Applicants show that increased fiber diameter leads to increased maximum tensile strength of the scaffold. PBGMH4 scaffold show promising potential as drug release carriers due to the slow and steadily release of MH. However, Applicants note that the drug release capacity may decrease over time. This decrease may be due to reduced specific surface area caused by fiber merging over time. Nevertheless, the present studies indicate that the drug release capacity can be restored by physically pulling apart merged fibers after tensile testing, emphasizing the crucial role of scaffold specific surface area in controlling drug release kinetics. Also, this determination is important for defining the efficacious period of the novel drug delivery system.

### Scaffold

Disclosed herein is a drug delivery system comprising a scaffold device composed of a composition of polypeptide-based electrospun fibers and the neuroprotective antibiotic MH. The disclosed systems, devices, and compositions may be useful in treating various diseases and pathological conditions of multiple physiological systems, as well as for neural tissue engineering.

The disclosed scaffolds may be comprised of various polymeric compounds. In various embodiments, the compounds may be biopolymers, for example polypeptide-based compounds. In various embodiments, the polypeptides may be comprised of one or more neuronal stimulants, for example glutamate and glutamic acid. In many embodiments the polypeptides may be selected from, poly(γ-benzyl-L-glutamate)(PBG) and its hydrolyzed copolymer (poly(γ-benzylglutamate) 80-r-(γ-glutamic acid)20)(PBGA), which contain neuronal stimulant glutamate and glutamic acid.

The disclosed scaffolds may be fabricated to include one or more bioactive compounds. In many embodiments, the scaffolds may be formed of a mesh of fibers. In some embodiments, the fibers may be comprised of biopolymer compounds and and one or more compounds with various beneficial activities, for example compounds with neurotrophic and/or neuroprotective activity. In one embodiment, the active compound may be minocycline hydrochloride (MH). In some embodiments, the active compound may be a peptide growth factor, for example nerve growth factor (NGF). The active compounds may be added before or after fiber formation. In some embodiments, mixing the polymer and active compound prior to fiber formation, may aid in embedding the active compound in the fiber and conferring the scaffold with enhanced release characteristics. In various embodiments, the active compound may comprise between about 0.01% to about 10% by weight of the fiber, for example more than about 0.01%, 0.5%, 1.0%, 1.5%, 2.0%, 2.5%, 3.0%, 3.5%, 4.0%, 4.5%, 5%, 6%, 7%, 8%, or 9%, and less than about 10%, 9%, 8%, 7%, 6%, 5.5%, 5.0%, 4.5%, 4.0%, 3.5%, 3.0%, 2.5%, 2.0%, 1.5%, 1.0%, and 0.5% by weight.

### Fabricating Fibers and Scaffold

The monomeric and polymeric materials may be synthesized by various methods. In one embodiment, according to the previous report and illustrate below in brief. Monomer may be synthesized and then polymerized. In several embodiments, the monomer is γ-benzyl-L-glutamaic acid N-carboxyanhydride (BGNCA), and may be synthesized by combining L-glutamic acid γ-benzyl ester, anhydrous THF, and propylene oxide. In other embodiments, the L-glutamate acid γ-benzyl ester may be combined with anhydrous ethyl acetate and triphosgene. n-hexane may be added to promote precipitation and the compound allowed to crystallize BGNCA may have a molecular weight of 263.25 g/mol. Other protein moieties or peptides or endogenous amino acids may be useful as discussed in Klimek, et al., Proteins and Peptides as Important Modifiers of the Polymer Scaffolds for Tissue Engineering Applications—A Review, Polymers (Basel). 2020 April; 12 (4): 844.

Polymerization may be achieved by various methods. In one embodiment, polymerization may be initiated after addition of benzene to disperse the monomeric BGNCA. In some embodiments, sodium methoxide may be used as an initiator, and PBGA formed by reacting PBG with trifluoroacetic acid and HBr. In many embodiments, the molecular mass of the polymers may be from about 100 to about 500 kDa, for example greater than about 100, 150, 200, 250, 300, 350, 400, or 450 and less than about 500, 450, 400, 350, 300, 250, 200, or 150 kDa.

Various methods may be used to form fibers from the disclosed polymeric compounds. In many embodiments, the fibers are formed via electro spinning process as described in Su, A.-J.A., et al., Fibrous polypeptide based bioscaffold delivery of minocycline hydrochloride for nerve regeneration. Materials Chemistry and Physics, 2023. 305: p. 127974., and Chen, T. C., et al., Polybenzyl Glutamate Biocompatible Scaffold Promotes the Efficiency of Retinal Differentiation toward Retinal Ganglion Cell Lineage from Human-Induced Pluripotent Stem Cells. Int J Mol Sci, 2019. 20 (1). In many embodiments, the one or more active compound may be dispersed in the polymer prior to fiber formation, while in other embodiments, the one or more active compounds are added after the fiber is formed. In many embodiments, the fibers form the disclosed scaffold, mat, or felt with an orientation of fibers. In some embodiments, orientation of the fibers may be aided by rotating a collector at rate of about 1000 rpm or higher. In some embodiments, the presence of a peptide linkage in the polymer chains may increase their rigidity and aid in orienting the resulting fibers.

### Characteristics of Disclosed Scaffold

The disclosed scaffolds may be of various thickness. In many embodiments, the disclosed scaffold thickness may range from about 10 μm to about 150 μm, for example about 60 micron, or greater than about 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, or more and less than about 200 μm, 150 μm, 140 μm, 130 μm, 120 μm, 110 μm, 100 μm, 90 μm, 80 μm, 70 μm, 60 μm, 50 μm, 40 μm, 30 μm, or 20 μm. The porosity of the disclosed scaffolds may range from about 10% to 99%, for one example about 90%, or greater than about 30%, 40%, 50%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%. The porosity of the scaffold may aid in nutrients flow in and around the scaffold as well as flow of active compounds. The fiber diameter may be of various sized, in many embodiments, the diameter may range from about 1000 μm to about 1000 nm, for one about 500 nm or more, in other embodiments greater than about 1 nm, 10 nm, 50 nm, 100 nm, 150 nm, 200 nm, 250 nm, 300 nm, 350 nm, 400 nm, 450 nm, 500 nm, 550 nm, 600 nm, 700 nm, 800 nm, or 900 nm, and less than about 1000 nm, 900 nm, 800 nm, 700 nm, 600 nm, 500 nm, 400 nm, 300 nm, 200 nm, 100 nm, or 50 nm. The fiber diameter may be useful in aiding cell adhesion to the scaffold—for measuring by SEM imaging of fibers as shown in Appendix Figure S5. In many embodiments, the fiber diameter may be varied depending on the polymer type, concentration, conductivity, presence, type, and concentration of active compound, and viscosity of the polymer and polymer+active compound(s) solution. In some embodiments, for example a salt, can increase the conductivity of the polymer solution, making the fiber easier to form by electro spinning. This may, in turn reduce the fiber diameter.

### Release Kinetics

The disclosed scaffolds may release active compounds from the fibers slowly. In most embodiments, less than about 85% of the active compound is released in the first 24 hours, for example less than about 81.20%. In many embodiments, the disclosed scaffolds may release less than about 30% in the first 24 hours, for example more than about 10%, 15%, 20%, 25%, 30%, 35%, or 40%, 45%, or 50%, and less than about 55%, 50%, 45%, 40%, 35%, 30%, 25%, 20%, or 15%. In many embodiments, after about 180 hours, the cumulative release of active compound may be greater than about 25%, 30%, 35%, 40%, 45%, 50%, 55%, 60%, 65%, or 70%, and less than about 75%, 70%, 65%, 60%, 55%, 50%, 45%, 40%, 35%, or 30%. In most embodiments, release of active compounds from the fiber scaffolds may generally follow the logarithm of natural law, for example Frick's Diffusion law, and may be modeled with one or more known diffusion models, for example the Higuchi model and Korsmeymer-Peppas model.

### Biocompatibility and Cell Support

The disclosed fiber scaffolds may display low or no cytotoxicity in the presence of mammalian cells, for example kidney or neuronal cells. In many embodiments, the disclosed more than about 90% of cells cultured in the presence of the disclosed scaffolds may survive, as evaluated by Live/Dead Assay see Appendix Figure S7, after 5-day of culturing, for example more than about 65%, 70%, 75%, 80%, 85%, 90%, or 95% and less than about 100%, 95%, 90%, 85%, 80%, 75%, or 70%. In some embodiments, the percentage of live cells after about 5-days in culture may be adversely or positively affected by the presence of one or more active compounds, and may change survival (relative to fibers without one or more active compounds) by more than about 1%, 2%, 3%, 4%, 5%, 6%, 7%, 8%, 9%, or 10% and less than about 15%, 10%, 9%, 8%, 7%, 6%, 5%, 4%, 3%, or 2%.

Cell differentiation and/or process outgrowth may be positively affected by the disclosed novel fiber scaffolds. In many embodiments, process outgrowth, for example neurite outgrowth and length, are positively affected. In these embodiments, the length of neurites from nerve growth factor-induced (NGF-induced) neuronal cells was greater in the presence of the disclosed polypeptide fibers compared to other polymer fibers, without or with one or more additional active compounds.

The disclosed fiber scaffolds may aid in directing axonal axonal regeneration and/or growth of neuronal cells. In many embodiments, The neurite outgrowth orientation was analyzed and plotted by polar chart (supporting information, see Appendix Figure S9processes such as neurites may be more aligned when grown on or near the presently disclosed PBG and PBGA fiber scaffolds. In most cases, this directionality may aid in restoring disrupted nervous systems. The degree of orientation of the disclosed fibers and outgrowth processes, such as neurites, may be high. In many embodiments, the degree of orientation in the disclosed scaffold may be defined as the percentage of fibers or of neurites aligned in the degree of 0+15° and 180+15°. In many embodiments, the degree of orientation is greater than about 50%, for example greater than about 25%, 30%, 35%, 40%, 45%, 50%, 55%, 60%, 70%, 80%, 90%, or 95% and less than about 100%, 90%, 80%, 70%, 60%, 50%, 40%, or 35%.

The disclosed fibers and scaffolds may display superior storage stability. In many embodiments, fibers comprising one or more active compounds may display enhanced stability compared with scaffolds comprising fibers without one or more active compounds. In some embodiments, for example where the active compounds(s) comprise one or more functional groups, electrostatic repulsion between fibers may be increased which may prevent (or retard) merging of individual fibers. This may enhance the duration of structural stability and utility of the disclosed scaffold compared with scaffolds comprising fibers of different fibers.

The disclosed scaffolds may possess enhanced mechanical strength compared with scaffolds comprising fibers of different fibers. In many embodiments, the presently described fiber scaffolds may display tensile strength of about 5 to about 10 MPa when fabricated, which may increase by 50% or more over time, for example over 12 months.

Applicants claim—

1. A system for supporting mammalian cells is disclosed, comprising:
a scaffold comprising a plurality of fibers, wherein the majority of fibers are oriented
substantially parallel; and
at least one active compound.

2. The system of claim 1, wherein at least one active compound is a growth factor.

3. The system of claim 1 or claim 2, comprising a neuronal cell and the at least one active compound is nerve growth factor.

4. The system of any one of claims 1-3, wherein the fibers comprise one or more of poly(γ-benzyl-L-glutamate)(PBG), hydrolyzed copolymer (poly(γ-benzylglutamate) 80-r-(γ-glutamic acid)20)(PBGA), and minocycline hydrochloride (MH).

5. A composition comprising:
a polymer comprising at least one trophic factor, and
at least one active compound.

6. The composition of claim 5, the polymer selected from poly(γ-benzyl-L-glutamate)(PBG), and hydrolyzed copolymer (poly(γ-benzylglutamate) 80-r-(γ-glutamic acid)20)(PBGA); and the at least one active compound is minocycline hydrochloride (MH).

7. The composition of claim 5 or claim 6 fabricated as a fiber.

8. A method of forming a scaffold for growth or support of a mammalian cell, comprising:
combining a polymer selected from poly(γ-benzyl-L-glutamate)(PBG), and hydrolyzed copolymer (poly(γ-benzylglutamate) 80-r-(γ-glutamic acid)20)(PBGA), with at least one active compound, wherein the active compound is minocycline hydrochloride (MH) to form a polymer composition;
forming a fiber from the polymer composition; and
arranging the fiber to create a scaffold.

9. The method of claim 8, wherein the forming and arranging are accomplished using electro spinning.

10. The method of claim 8 or 9, wherein the concentration of the active compound is less than about 5% by weight.

11. A method of treating a peripheral nerve injury, comprising:
identifying a mammalian subject having an injury to a cell or tissue;
surrounding the injured cell or tissue with the system of any one of claims 1-4, or the composition of any one of claims 5-7.

12. The method of claim 11, wherein the injury is to a sciatic nerve.

13. A device comprising:
fiber scaffold comprising;
   a polymer; and
   an active compound.

14. The device of claim 13, wherein the polymer is selected from poly(γ-benzyl-L-glutamate)(PBG), and poly(γ-benzylglutamate) 80-r-(γ-glutamic acid) 20 (PBGA).

15. The device of claim 13 or claim 14, wherein the polymer PBGA.

16. The device of any one of claims 13-15, wherein the active compound is minocycline hydrochloride (MH).

17. The device of any one of claims 13-16, wherein the active compound is less than about 5% by weight of the fiber.

18. The device of any one of claims 13-17, wherein release of the active compound from the fiber does not show two-phase kinetics after 24 hours.

19. The device of any one of claims 13-18, wherein at least 50% of the fibers are oriented in the same or the opposite direction.

20. The device of any one of claims 13-19, for use in treating a peripheral or central nervous system cell or tissue.

### Examples

### Example 1—Characterization of PBG and PBGA Scaffolds

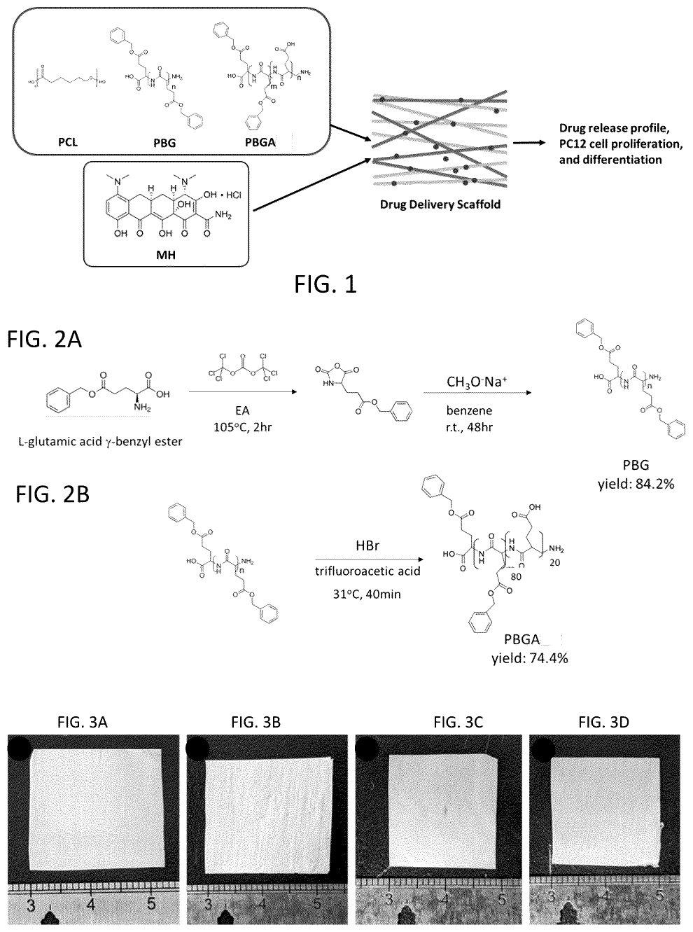

Three kinds of 3D scaffolds with aligned fibers were made from PCL, PBG, and PBGA respectively using electrospinning process. For each polymer, four kinds of samples were prepared that were incorporated with either 2 wt. %, 4 wt. %, 6 wt. % MH (as used herein wt. % is wt/v % MH) or without any MH, respectively. PCL was used as a control scaffold. The materials properties and release profile for each scaffold (n=12) were investigated. PC12 cell viability and proliferation on each of these scaffolds was studied and analyzed. FIG. 1 shows the schematic diagram of this drug delivery system.

### Characterization of Polymeric Materials for Fibrous Scaffolds

The polymeric materials, PBG and PBGA, were synthesized according to the reactions shown in FIGS. 2A and 2B. The synthetic yield for PBG was about 84.2%; partially hydrolyzed PBG (PBGA containing 20 molar % of glutamic acid in PBG) was produced at a yield of about 74.4%. GPC was used to characterize the molecular weight of PBG with DMF as a mobile phase. The average molecular weight of PBG was controlled between about 200 and 300 kDa. This size range aided scaffold processing and produced quality fibers. The results are summarized in Table 1 indicating the synthesis of polymer is reproducible. The chemical structures of PBG and PBGA were confirmed by NMR and FTIR (supporting information, at Appendix Figures S1 and S2, Table S1). These results confirmed the successful synthesis of PBG and PBGA.

TABLE 1
Properties of polymeric material of PBG including number
average molecular weight (Mw), weight average molecular
weight (Mw), polydispersity, and yield.

| Sample number | Mn (kDa) | Mw (kDa) | Polydispersity | Yield (%) |
|---|---|---|---|---|
| 1 | 249.2 | 251.6 | 1.01 | 86.7 |
| 2 | 199.9 | 226.2 | 1.13 | 83.5 |
| 3 | 171.2 | 219.7 | 1.28 | 82.4 |

### Characterization of Scaffolds

Photographic images of PBGA scaffold with different MH concentration are shown in FIGS. 3A-3D. As shown therein, MH is distributed in the scaffolds as indicated by the color change from white to tinted yellow, with the yellow becoming more intense as the concentration of drug increased. The drug MH was incorporated into the polymer in the solution state by mixing them in a mixed solvent of DMAc and THE overnight. The solution compositions are shown in Appendix Table S2. Then the solution was used to fabricate fibers via electro spinning process. Thus, the drug is molecularly dispersed in the polymer and its amount will not be affected by the orientation of fibers property. The result indicates the MH can be evenly distributed in the fibers which is helpful to interpret the drug release mechanism using good reproducible samples. It was reported the PCL electrospinning fibers can be aligned by rotating collector at rate of more than 1000 rpm. The morphology of electrospun fibers was studied by SEM as shown in Appendix Figure S4 (supporting information). The fibers of the PBG and PBGA polypeptides are straighter and more aligned than those of PCL fibers. Without wishing to be limited by theory, Applicants hypothesized that the polypeptides of PBG and PBGA contain aromatic benzyl group in the repeating unit and hydrogen bonds between peptide linkage of polymer chains making them relatively rigid and easy to align. On the other hand, PCL consists of aliphatic repeating unit of caprolactone and no hydrogen bonds between ester linkage of polymer chains which makes PCL flexible and curvaceous. Thus, PCL is more difficult to align than PBG and PBGA. The present results are consistent with the literature report that indicates the Young's modulus of PBG is 4 times higher than that of PCL. The thickness of scaffolds used in the cell culture experiment was in the range of 60 micron with the porosity in the range of 90%. Thus, the scaffolds were in a 3D porous structure to facilitate nutrients flow in the cell culture experiments. The fiber diameter was controlled to be larger than 500 nm for the ease of cell adhesion on the substrate and it was measured by SEM imaging of fibers as shown in Appendix Figure S5. The fiber diameter depends on the polymer type, concentration, conductivity and viscosity of the polymer solution. The effects of MH addition and incorporation upon fiber diameter was investigated. Since MH is a salt, its addition can increase the conductivity of the polymer solution, making the fiber easier to spin and thus reducing fiber diameter. The extent of dimension reduction was decreased by increasing the polarity of the polymer. Therefore, the PBGA polymer exhibited the least amount of reduction in diameter compared to PCL or PBG. The diameter of PCL increased when the concentration of MH was increased up to 6 wt. %. This might be due to an increase in viscosity, which overcomes the conductivity effect; which subsequently increased the dimension of PCL fibers. The alignment of the fibers in the scaffolds was evaluated and results are discussed in later sections below.

Drug Release from Scaffolds

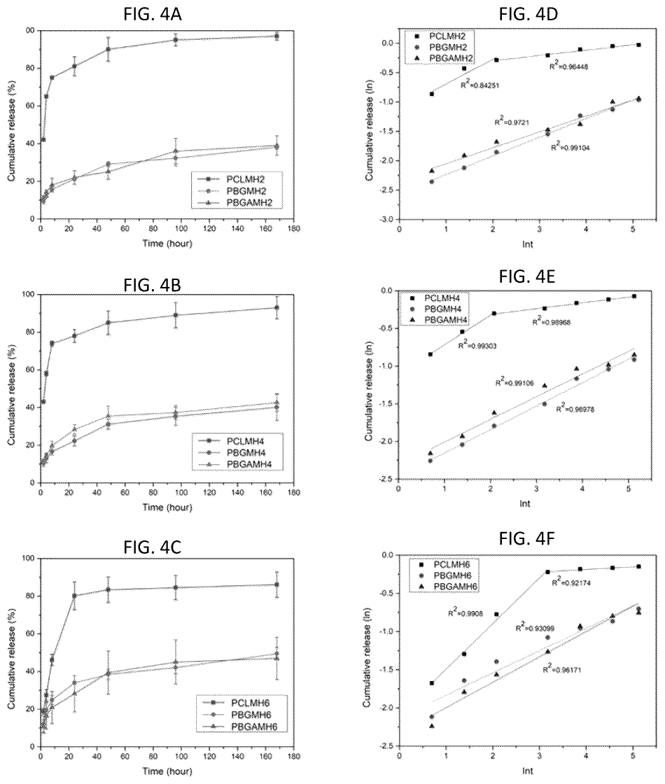

During drug release from scaffolds, a cascade of partitioning and diffusion with fibers and dissolution occurs while immersed in PBS. To understand the mechanism behind observed release profiles, known kinetic models of mathematics were used. The drug release profile from electrospun fibers may be dominated by drug diffusion and material degradation. The release profiles from polymer scaffold with various concentrations of MH (2, 4, 6 wt. %) within 7 days are shown in FIG. 4. For PCL scaffold, there was a burst of release in the first 24 hours, releasing 81.20%, 78.98%, and 80.14% of the drug in 2 wt. %, 4 wt. %, and 6 wt. % MH scaffold, respectively. On the other hand, taking PBGMH4 and PBGAMH4 for instance, the scaffolds only released 22.21% and 28.32% of MH in the first 24 hours respectively. The results indicate the drug release from PCL scaffolds are two functions (FIG. 4D black line) due to the burst release of drug initially then follows the logarithm of natural law (FIG. 4A, 4B, 4C red lines). The drug release of polypeptide scaffolds follows the logarithm of natural law (FIG. 4A, 4B, 4C blue and green lines), one function equation can be derived (FIG. 4D, 4E, 4F blue and red lines). The results are very reproducible as indicated in three sets of data of PBG based and PBGA based scaffolds that are compared with PCL scaffolds by varying the loading of the MH drug. The hydrophilicity of the polymers was measured by their water contact angle of dense films as shown in Appendix Figure S6 (supporting information), the average contact angle was 75°, 82°, 76° for PCL, PBG, and PBGA respectively. In the aqueous culture solution, the water-soluble MH is released more readily from the more hydrophilic polymer, PCL. Although the hydrophilicity of PBGA is close to that of PCL, MH release is slower for PBGA by the increased number of strong hydrogen bonds between PBGA and MH compared to PCL (amide chain versus ester chain).

The release profiles were fitted with two mathematical kinetic models, Higuchi model and Korsmeymer-Peppas model (supporting information, Appendix Table S3). The drug release profile can be affected by drug diffusion and carrier degradation. The mechanism of drug release kinetics has been explained by several mathematical models, including zero-order equation, first-order equation, Higuchi equation, and Korsmeyer-Peppas equation. Since the drug release profiles show a faster release in the first 24 hours and a relatively sustained release thereafter, Korsmeyer-Peppas equation is a more appropriate model to fit the release profile in this study (FIGS. 4D, 4E, and 4F) with n<0.45. The n value of Korsmeyer-Peppas equation can determine the release profile into Fick's diffusion (n<0.45) or non-Fick's diffusion (0.45<n<0.89). The former indicates the release profile follows Fick's diffusion, while the latter indicates the release profile is non-Fick's diffuse n, or skeleton degradation. Our previous work demonstrated that the PCL, PBG, and PBGA scaffolds barley degrade in the release duration of 7 days. These results clearly indicate that the MH released from scaffolds examined in this study follow Fick's diffusion law.

### PC12 Cell Viability on Scaffold

The cytotoxicity of each scaffold was evaluated by Live/Dead Assay (Supporting information, Appendix Figure S7). The left figure of Figure shows that there were more live cells on PBG and PBGA scaffolds than PCL scaffolds after 5-day culturing, denoting that the novel polypeptide-based scaffolds are less cytotoxic. Moreover, the number of live cells were more numerous on scaffolds with 2 wt. % and 4 wt. % of MH but, decreased as the drug concentration reached 6 wt. %. After 5 days of culturing, the live/dead ratio of cells on PBG scaffolds with 2 wt. % and 4 wt. % of MH were above 90%, indicating a high degree of biocompatibility.

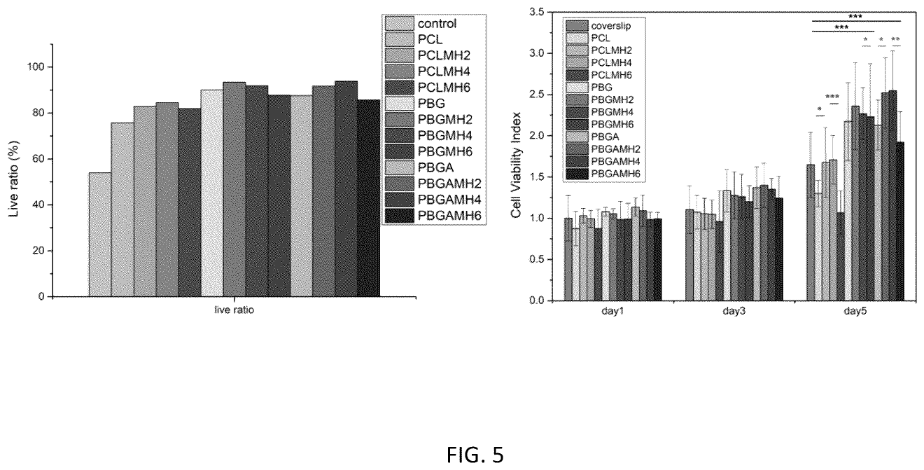

The right graph of FIG. 5 shows the results of cell viability over day 1, day 3, and day 5 for each scaffold. All the materials containing different MH concentration present increased cell viability index over 5 days of culturing as compared to respective bare scaffold, indicating all the materials are biocompatible. The cell viability was much higher on PBG and PBGA scaffolds than PCL scaffolds and coverslip control group, indicating better cell adhesion and proliferation on the polypeptide scaffolds. When considering the addition of MH, the cell viability increased linearly for each scaffold. The effect of MH concentration upon viability was best between 0 wt. % to 2 wt. % and 4 wt. %, demonstrating that adding MH promoted cell proliferation. Surprisingly, when MH concentration increased to 6 wt. %, the cell viability index decreases, showing high concentration of MH inhibited the proliferation.

Among all the tested materials, cells cultured on PBGA scaffold had the highest cell viability. This may be a result of engineering a polypeptide backbone using the neuron transmitter-like glutamic acid moiety. The PBG scaffold contains benzyl glutamate repeating unit which is different from glutamic acid and is less effective for neuronal signaling than glutamic acid. This may explain why PBG shows slightly lower cell viability character than PBGA. Regardless, both the polypeptide PBG and PBGA exhibit better biocompatibility than that of PCL due to their biomimetic structures.

To investigate the effect of MH concentration on cellular activity, MH concentration after 24 hours in vitro was estimated using the fitted release kinetic data above. It is assumed that both release test and cell tests were under similar condition, suggesting the release results can be correlated with the results of cell viability and differentiation. Literature reported that PC12 cells maintain good cell viability and differentiation when the concentration of MH was at 30 μM. We found PC12 viability increased incrementally with MH concentration up to 4 wt. % but decreased in cell viability at 6 wt. %. After 24 hours in vitro experiments, the scaffold contained 4 wt. % MH is estimated from the kinetic model to be 38.3 μM for PBGMH4, 49.5 μM for PBGAMH4. For the scaffold contained 6 wt. % MH, the estimated value is 94.8 M for PBGMH6 and 78.6 μM for PBGAMH6 (supporting information, Appendix Table S4). Thus, without wishing to be limited by theory, Applicants speculate that the high concentration of MH may induce cytotoxicity, and thus lower the cell viability. To sum up, both Alamar blue test and live and death assay performance show the PBGA scaffold with 4 wt. % MH exhibited the highest cell viability.

### Additional Cell Lines Tested 551W and HEK293.

### PC12 Cell Differentiation and Neurite Growth on Scaffold

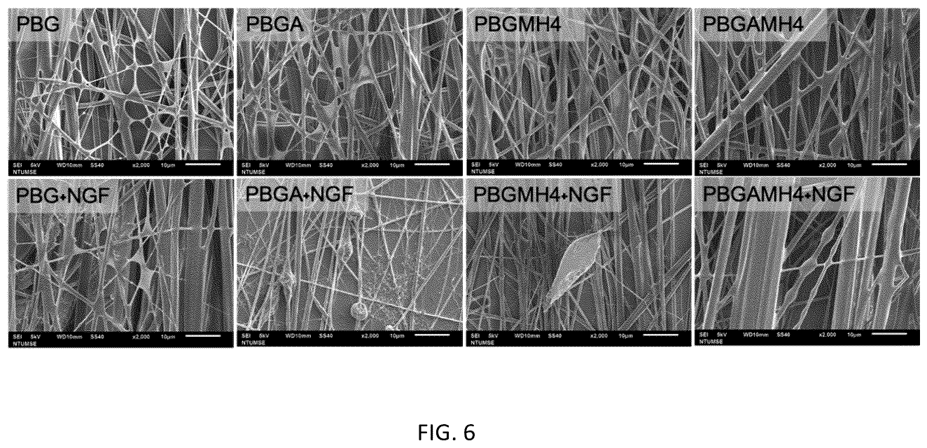

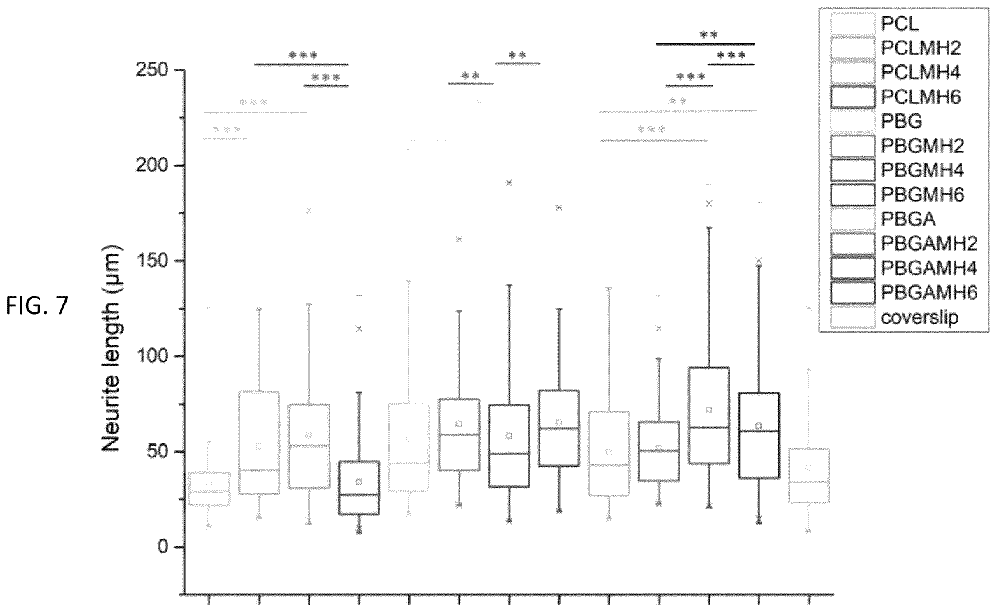

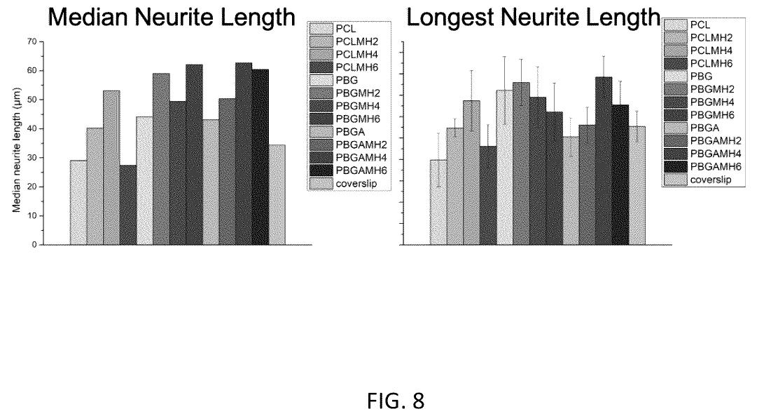

Cell differentiation and neurite outgrowth were investigated by SEM (FIG. 6) and cytoskeletal staining of PC12 cells (supporting information, Appendix Figure S8). PC12 cells were stimulated by exogenous NGF for five days and neurite elongation was assessed. To quantify the results of neurite formation and extension, neurite lengths were measured by software ImageJ and statistically analyzed (FIG. 7). The results of cell differentiation are similar as those of cell toxicity and cell viability. The effects of material type of scaffolds on cell differentiation demonstrate that PC12 have longer neurites on PBG and PBGA scaffolds than PCL scaffolds. The neurite lengths increased with MH increment up to 4 wt. % but decreased when the MH concentration exceeded 4 wt. %. The neurite lengths of PC12 cells were significantly longer on PBGA scaffold with 4 wt. % of MH than other groups. Moreover, if comparing median and longest neurite length among different scaffolds with various MH concentration, the same conclusions is found. That is, the cell culture on PBGA4MH produced longest and highest median neurite length (FIG. 8). The trend of neurite length follows the results of cell viability.

Misdirected axonal regeneration and the failure of long range axonal regeneration challenge the restoration of disrupted nervous systems. The neurite outgrowth orientation was analyzed and plotted by polar chart (supporting information, Appendix Figure S9). The neurites are more aligned on PBG and PBGA scaffolds with various concentration of MH than PCL scaffolds and the control group coverslips. This directionality may result from an increased guidance by aligned fibers in polypeptide scaffolds (Appendix Figure S10). Increased alignment of neurites extending on PBG and PBGA suggests that neurites can extend along the polypeptide scaffolds successfully, and the addition of MH does not affect the direction of neurite on the fibers.

Comparing the Orientation of Neurite Extension with the Orientation Fibers in Scaffold

Normal neurophysiology requires developing neurons to target and form appropriate synapses with target cells, often over great distances (such as from retinal ganglion cells to the brain cortex). Neurons communicate using neurotransmitters that are synthesized and then transported from soma to the axon tip. As such, restoration of compromised nervous systems and accompanying cytoarchitecture can be enhanced by the engineering the alignment of neurite outgrowth. This alignment and directionality can be controlled by the orientation of fibers in the scaffold. The degree of orientation of fibers in the disclosed scaffolds was determined by software ImageJ using polar chart derived from SEM photos of scaffolds shown in Appendix Figure S4. The results are shown in Appendix Figure S10. The degree of orientation of fibers and neurites in scaffold are defined as the percentage of fibers or neurites aligned in the degree of 0+15° and 180+15° of the images. The results are summarized in Table 3. The degree of orientation of fibers in each scaffold was all above 50%, indicating that fibers were well aligned. The result of neurite orientation also demonstrates that the outgrowth of neurite extends in specific direction, especially for PBG scaffold and PBGA scaffold, demonstrating that the polypeptide scaffolds with aligned fibers could successfully guide the outgrowth of neurites.

TABLE 3
Degree of orientation of fibers and neurite
outgrowth on different scaffolds.

| Material | % Orientation of fibers^{a} | % Orientation of neurites^{b, c} |
|---|---|---|
| PCL | 76.92 | 49.41 |
| PCLMH2 | 67.92 | 29.05 |
| PCLMH4 | 66.67 | 30.84 |
| PCLMH6 | 57.14 | 36.94 |
| PBG | 63.64 | 67.74 |
| PBGMH2 | 73.68 | 80.02 |
| PBGMH4 | 60.87 | 80.52 |
| PBGMH6 | 72.50 | 79.52 |
| PBGA | 72.22 | 51.56 |
| PBGAMH2 | 82.35 | 64.82 |
| PBGAMH4 | 71.05 | 71.73 |
| PBGAMH6 | 63.13 | 77.12 |

^{a}Degree of orientation of fibers is defined by the percentage of fibers oriented in the range of 0 ± 15° and 180 ± 15° of the polar images of fibers. Showing the fibers of scaffolds are aligned well.
^{b}Degree of orientation of neurite outgrowth on different scaffold. Showing the neurites grown along the orientation of fibers in scaffolds.
^{c}Degree of orientation of neurite outgrowth on coverslip is 24.99%.

### Example 2—Stability of Scaffold

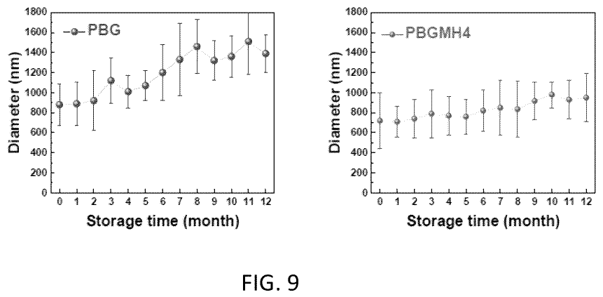

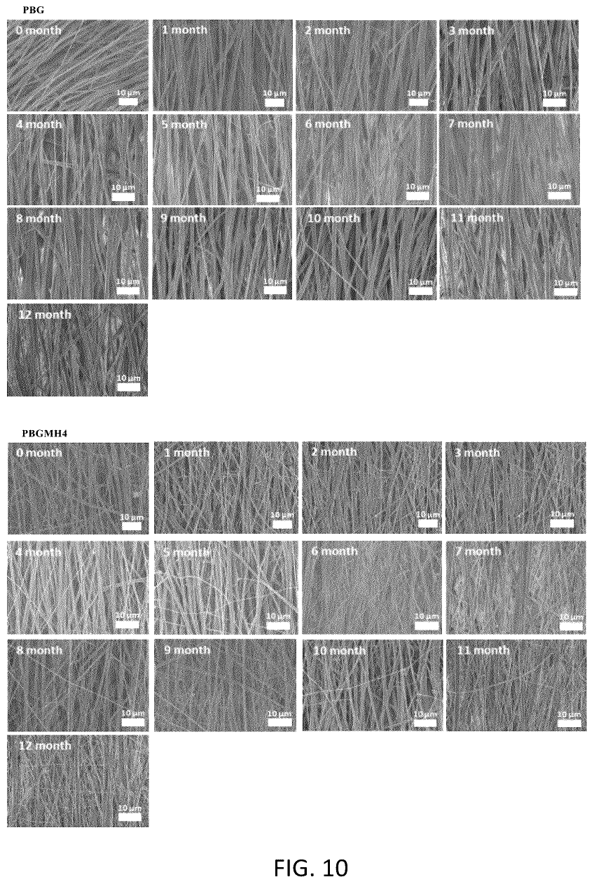

In order to investigate the storage stability of PBG and PBGMH4 aligned fiber scaffolds felts prepared via electrospinning, the scaffolds were stored under ambient conditions. Variation in fiber diameter on the top side of the felts was monitored over a storage period of 12 months, as shown in FIG. 9, and summarized the diameter data in Table S1. Detailed SEM top view images of the fiber scaffolds for each month are available in FIG. 10. The initial fiber diameters of PBG and PBGMH4 scaffold were 880 nm and 720 nm, respectively. The smaller size of the PBGMH4 fiber diameter was attributed, by Applicant, to the presence of MH, which acts as a salt. MH enhances conductivity of the PBG solution and facilitates easier fiber formation during the initial high-voltage electrospinning process. This charge effect leads to a size difference of 22.2% [=(880-720)/720] between PBG and PBGMH4 scaffolds.

TABLE 4
Detailed top side fiber diameter variation in PBG and PBGMH4
scaffold felts over a storage period of 12 months.

| | |
|---|---|
| Sample | Fiber diameter (nm) |

| Storage time | PBG | PBGMH4 |
|---|---|---|
| Fresh | 880 ± 210 | 720 ± 280 |

| | | | |
|---|---|---|---|
| 1 | month | 890 ± 220 | 710 ± 150 |
| 2 | month | 920 ± 300 | 740 ± 190 |
| 3 | month | 1120 ± 230 | 790 ± 240 |
| 4 | month | 1010 ± 160 | 770 ± 190 |
| 5 | month | 1070 ± 150 | 760 ± 170 |
| 6 | month | 1200 ± 280 | 820 ± 210 |
| 7 | month | 1330 ± 360 | 850 ± 270 |
| 8 | month | 1466 ± 270 | 835 ± 280 |
| 9 | month | 1320 ± 200 | 917 ± 190 |
| 10 | month | 1360 ± 210 | 980 ± 130 |
| 11 | month | 1510 ± 330 | 930 ± 190 |
| 12 | month | 1390 ± 190 | 950 ± 240 |

As the storage time increased, Applicants noted that the fiber diameter of the PBG scaffold increased. Under ambient storage conditions, the fiber diameter increased by 58% [=(1390-880)/880] over the 12 months storage period. On the other hand, the fiber diameter of PBGMH4 scaffold increased more slowly, with only a 32% [=(950-720)/720] increase after 12 months of storage. Without wishing to be limited by theory, Applicants hypothesized that on reason for the difference in fiber diameter increment is that with prolonged storage time, the weak van der Waals forces between PBG polymer chains gradually cause them to merge. In contrast, the presence of MH in the PBGMH4 scaffold, along with minocycline carrying NH3+functional groups, may lead to electrostatic repulsion between fibers preventing (or slowing) merging. These results suggest that the PBGMH4 scaffold may possess superior storage stability compared to that of PBG alone.

As a result of extended storage, the fiber diameter on the top side of PBG scaffold felt had significantly increased. Consequently, the SEM cross-section images of PBG scaffolds felts just completed by electrospinning were compared with scaffolds that have undergone long-term 4877-0395-5421\1 storage, to observe whether there are changes in fiber diameter in the vertical direction of the scaffold felt. The SEM cross-section images are shown in FIGS. 11A-B. Analysis of these images reveals that the freshly completed electrospun PBG scaffold (FIG. 11A) has fine fiber diameter with minimal merging. However, after 9 months of storage, fibers are noticeably merging together (FIG. 11B). Without wishing to be limited by theory, this may be due to van der Waals forces, as well as the gravity effect on each fiber causing them to approach each other and sink downwards, ultimately leading to the collapse of the microstructure of PBG scaffold.

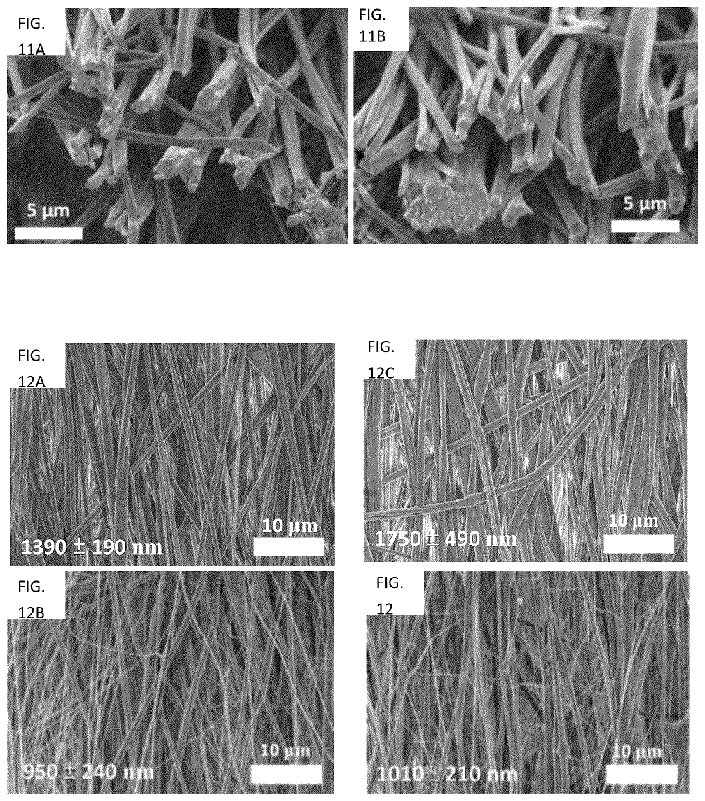

To verify the impact of gravity on the storage stability of scaffold felt, we also compared the top and bottom side SEM images of PBG and PBGMH4 scaffold felt, as shown in FIGS. 12A-12D. Observation of samples stored for 12 months reveals a significant variance in fiber diameter between the top and bottom sides of PBG scaffold felt, with a difference of 25.9% [=(1750-1390)/1390]. Without wishing to be limited by theory, this was hypothesized to indicate that beyond van der Waals forces, PBG scaffold felt is also influenced by gravity, causing fibers to merge together and resulting in thicker fibers on the bottom side compared to the top side. On the other hand, PBGMH4 scaffold felt shows only a 6.3% [=(1010-950)/950] difference in fiber diameter between the top and bottom sides. This suggested (again without limiting by theory) that while fibers in PBGMH4 scaffold felt are also affected by gravity and tend to merge, the electrostatic repulsion partially counteracts the effect of gravity, thus further demonstrating that PBGMH4 scaffold felt has better storage stability compared to PBG ones.

FIGS. 12A-D are SEM images of the (FIG. 12A) top side and (FIG. 12B) bottom side of PBG scaffold felts, as well as the (FIG. 12C) top side and (FIG. 12D) bottom side of PBGMH4 scaffold felts.

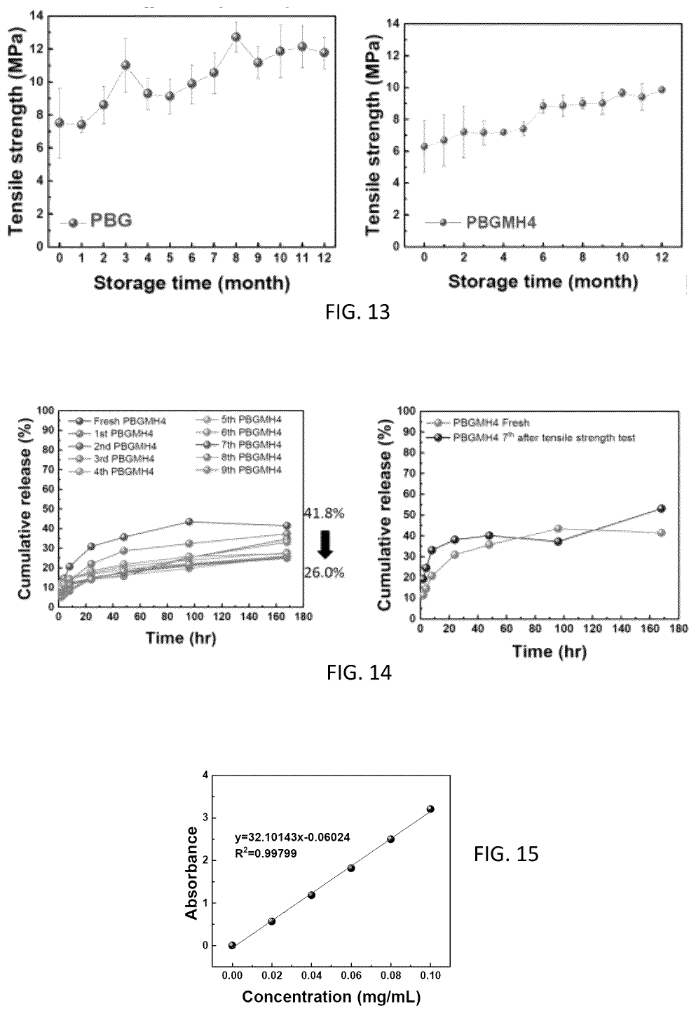

In the field of neural tissue engineering, the mechanical strength of scaffolds plays a significant role in cell proliferation and growth. Therefore, the variation of scaffold mechanical strength with storage time is a crucial issue when investigating scaffold stability. As noted above, the diameter of fibers increases with storage time, which may suggest a change in the mechanical properties of scaffold felts. The variation of maximum tensile strength over storage time for PBG and PBGMH4 scaffold felts was analyzed and the results shown in FIG. 13 and summarized in Table 5. The initial maximum tensile strengths of PBG and PBGMH4 scaffold felts are 7.53 MPa and 6.30 MPa, respectively. The slightly lower maximum tensile strength of PBGMH4 scaffold felts compared to PBG scaffolds is reasonable, given the smaller initial fiber diameter of PBGMH4 scaffold felts. With increasing storage time, both PBG and PBGMH4 scaffold felts exhibit a gradual increase in maximum tensile strength, consistent with the increase in fiber diameter. Ultimately, the maximum tensile strengths of PBG and PBGMH4 scaffold felts reach 11.78 MPa and 9.86 MPa, respectively.

TABLE 5
Detailed maximum tensile strength variation of PBG and PBGMH4
scaffold felts over a storage period of 12 months.

| | |
|---|---|
| Sample | Maximum tensile strength (MPa) |

| Storage time | PBG | PBGMH4 |
|---|---|---|
| Fresh | 7.53 ± 2.14 | 6.30 ± 1.64 |

| | | | |
|---|---|---|---|
| 1 | month | 7.42 ± 0.48 | 6.69 ± 1.59 |
| 2 | month | 8.62 ± 1.14 | 7.21 ± 1.64 |
| 3 | month | 11.02 ± 1.67 | 7.18 ± 0.76 |
| 4 | month | 9.30 ± 0.93 | 7.20 ± 0.16 |
| 5 | month | 9.14 ± 1.06 | 7.41 ± 0.45 |
| 6 | month | 9.90 ± 1.19 | 8.84 ± 0.43 |
| 7 | month | 10.57 ± 1.26 | 8.87 ± 0.67 |
| 8 | month | 12.73 ± 0.93 | 9.01 ± 0.36 |
| 9 | month | 11.17 ± 0.96 | 9.02 ± 0.70 |
| 10 | month | 11.87 ± 1.64 | 9.67 ± 0.24 |
| 11 | month | 12.15 ± 1.29 | 9.41 ± 0.83 |
| 12 | month | 11.78 ± 0.96 | 9.86 ± 0.24 |

FIG. 13. Maximum tensile strength variation of PBG and PBGMH4 scaffold felts over a storage period of 12 months.

The cumulative drug release profiles of PBGMH4 scaffolds with various storage times are depicted in FIG. 14 (left). PBGMH4 scaffold felts were stored under ambient conditions, with one sample taken out every month for a 7 days drug release test. The experimental results demonstrate that PBGMH4 scaffold can slowly and steadily release MH, indicating it is an excellent carrier for sustained drug release. However, despite the increase in fiber diameter and mechanical properties with storage time, the amount of cumulative drug release after 7 days gradually decreased. The freshly completed electrospun PBGMH4 scaffold felt exhibits a cumulative drug release of about 41.8% over 7 days, but after 9 months of storage, the cumulative drug release decreased to about 26.0%. Applicants hypothesized, without wishing to be limited, that since the fibers of both PBG and PBGMH4 scaffold are aligned without any crosslinkage, they may form a porous felt structure. With prolonged storage, fiber merging may lead to an increase in diameter, which may, in turn, resulting in a decrease in the porosity of PBGMH4 scaffold felt from 90% to 72% as measured by mercury porosimetry. This reduction in porosity may decrease the specific surface area of PBGMH4 scaffold, and lower its drug release capacity.

To verify if the decrease in specific surface area causes the decline in drug release capacity, we randomly selected a PBGMH4 scaffold stored for an extended period and conducted a drug release test after undergoing tensile testing (in this case, a sample stored for 7 months), as shown in FIG. 14 (right). Since above results indicate that the fiber merging is due to physical factors including gravity and van der Waals forces rather than forming chemical bond, the merged fibers were pulled apart after tensile testing and increasing the specific surface area again. Consequently, the drug release capacity is restored to a level similar to that of the freshly completed electrospun PBGMH4 scaffold felt. These results emphasize the crucial role of scaffold specific surface area in governing drug release kinetics.

### Example 3—Materials and Methods

### Chemicals and Materials

Chemicals used for the synthesis of PBG, PBGA include L-glutamate acid-benzyl ester (99% purity; Flurochem), triphosgene (98% purity; Sigma), sodium (99% purity; Sigma), dichloroacetic acid (99% purity; Acros), 33 wt. % HBr in acetic acid (99% purity; Acros), sodium hydroxide (98% purity; Acros), trifluoroacetic acid (99.5% purity; Acros), tetrahydrofuran (THF, 99% purity; Macron), dimethylformamides (DMF, 99.8% purity; Macron), N,N-dimethylacetamide (DMAc, 99.8% purity; Sigma). Anhydrous solvents of ethyl acetate (EA; 99.5% purity) and benzene (99.7% purity; Sigma) were prepared from the dehydration of the purchased ones by drying with 4A molecular sieves (4 to 8 mesh; Acros) and N2 purging overnight. Polycaprolactone (PCL, mol. wt. 282 KDa, PDI: 1.3, Sigma) was used as a control polymer in this study. Minocycline hydrochloride (MH, 98.0% purity, cat. #M2288, TCI) was purchased from TCI company.

Reagents used for cell culture include RPMI-1640 culture media (cat. #SH30011.02; Hyclone), nerve growth factor (NGF, cat. #N6009; Sigma), dimethyl sulfoxide (DMSO; cat. #SU01551000; Scharlau), trypsin-EDTA 10× (cat. #03-051-5B; Biological Industries), fetal bovine serum (FBS; cat. #04-001-1A), horse serum (HS, cat. #16050122; Gibco), antibiotic antimycotic solution 100× (PSA, cat #A5955; Sigma-Aldrich), PBS phosphate buffered saline solution (cat #BP3991, Fisher BioReagents). The solution of trypsin-EDTA 1× was prepared by diluting trypsin-EDTA 10× with PBS.

Chemicals for characterization of in vitro assays included Live/Dead viability/cytotoxicity kit (cat. #L3224; Molecular Probes), Alamar Blue cell toxicity assay (cat. #BUF102A; Serotec), bovine serum albumin (BSA; cat. #B4287; Sigma-Aldrich), formaldehyde (37 wt. %; cat. #50-00-0; ACROS Organics) and triton X-100 (cat. #X198-07; J.T. Baker). The stock solution of phalloidin was prepared by dissolving 0.1 mg phalloidin-TRITC in 1 mL DMSO (76.6 μM). The stock solution of 4′,6-diamidino-2-phenylindole (DAPI, cat. #D8417, Sigma-Aldrich) was prepared by dissolving DAPI in DI water (1 mg/mL), phalloidin-tetramethyl rhodamine B isothiocyanate (phalloidin-TRITC, cat. #P1951, Sigma-Aldrich).

### Nomenclature

Polycaprolactone is abbreviated as PCL. Two kinds of polypeptides are used in this study: poly(γ-benzyl-L-glutamate)(PBG) and poly(γ-benzyl-L-glutamate)-r-poly(γ-L-glutamic acid)(PBGA). The monomer of PBG, γ-benzyl-L-glutamate N-carboxyanhydride, is named as BGNCA. The minocycline hydrochloride is named as MH.

### Polymeric Material Synthesis and Characterization

The polymeric materials were synthesized according to the literature and briefly stated below. To synthesize BGNCA monomer, 6 grams of L-glutamate acid γ-benzyl ester was added with 180 mL of anhydrous ethyl acetate. 3.75 grams of triphosgene was added to the flux. The reaction proceeded for two hours at 105° C. under N₂, and the solution was cooled down to room temperature for 15-30 minutes after the reaction was completed.

The N-carboxyanhydrive (NCA) solution was poured into hexane, forming white BGNCA precipitate, and was stored at −20° C. for 1 day. The crude NCA was filtered and dissolved in the least amount of anhydrous ethyl acetate, and enough hexane was added into the NCA solution until a little white NCA crystal appeared. The crystallization process was continued at 4° C. overnight. The recrystallize process was repeated 3-4 times until the NCA formed shining white crystals, indicating that the NCA was pure enough to be polymerized. The purified NCA was dried at 40° C. in vacuum oven (Thermo Scientific 3608) for 1 day.

Three grams of BGNCA crystals were placed into a flask and dried under vacuum for 1 hour to remove moisture. 300 mL of benzene was transferred to the flask under N₂. Afterward, sodium methoxide solution was added to the flask all at once with stirring (molar ratio of initiator: monomer=1:100). The solution became clear and viscous after several minutes. The polymerization was conducted for 48 hours and then was precipitated in ether. After precipitation, the polymer was dried in a vacuum oven at 40° C. overnight.

To synthesize PBGA, 1 gram of PBG was put in a 50 mL flask and 25 mL of dichloroacetic acid (DCA) was added to the flask. The PBG was dissolved in the solvent overnight. After the polymer was fully dissolved, 760 μl of HBr solution (33 wt. % HBr in acetic acid) was added to the flask, reacting for 40 minutes at 31° C. Then the reactant was precipitated in ether with a minimal volume of methanol, stirring for 10 minutes. To purify, the precipitated polymer was re-dissolved in the least amount of THF and precipitated in ether several times. The polymer was dried in vacuum oven in 40° C., yielding PBGA.

The chemical structures of synthesized PBG and PBGA were characterized by spectroscopies of NMR (Brucker; DPX400) and ATR-FTIR (Perkin Elmer; Spectrum 100), respectively. 5-10% (w/v) of polymer was dissolved in trifluoroacetic acid d-solvent for ¹HNMR analysis. The polymer films of PBG and PBGA used for IR measuring were made by drop casting of the tetrahydrofuran (THF) solution. The NMR and IR spectra of products are shown in supporting information (see Appendix Figure S1, Figure S2, and Table S1).

The molecular weight of PBG was determined by gel permeation chromatography (GPC) with 0.5% (w/v) polymer solution in dimethylformamide (DMF). The average molecular weight of the PBG was controlled in the range of 200K to 300K Dalton for the ease of electrospinning process and good quality of the fibers. The results are listed and discussed below.

### Scaffold Fabrication and Characterization

The electrospinning process typically involved for the polymer solutions of PCL, PBG, and PBGA with 2 wt. %, 4 wt. %, and 6 wt. % of MH was prepared respectively using co-solvent of THF and DMAc overnight. A grounded, rotating collector was placed 15 cm away from the needle tip. For thick fibrous samples (scaffold mat) used in drug release test and mechanical properties test, the collector was first wrapped with aluminum foil and fibers were collected on it for about 20 minutes; then the mat was covered with wax paper, which made the scaffold mat easier to be removed. For cell test, aluminum foil was wrapped on the collector, and glass coverslips were pasted on the aluminum foil to collect fibers. The preparation of solution and electrospinning parameters were listed in supporting information Appendix Table S2. The thickness of mat was controlled in 50-70 micron by the electro spinning time of the fiber fabrication. The thickness was determined by micrometer. The porosity of the mat was controlled in 80-90% by varying the spinning time of fiber fabrication. The porosity was determined by Hg porosimetry.

### Drug Release

Thick scaffold mats were removed from the wax paper and cut into 2×2 cm² pieces. For each test scaffold pieces weighing at least 15 mg are placed in a sample bottle containing 10 mL PBS. For each time point (2, 4, 8, 24, 48, 96, and 128 hours), 1 mL PBS supernatant was removed from the sample bottle to measure the released MH, and an equal volume of 1 mL fresh PBS was added to the bottle. The concentration of MH released from the polymer scaffold was measured by UV-Vis spectrometer with a calibration curve made at wavelength 245 nm. Each test was repeated three times. The cumulative release was calculated by the following equation.

$$Cumulative release ( \% ) = \frac{weight of the drug released}{weight of the total drug} \times 100$$

### Kinetic Modeling for Drug Release

The drug release profiles were fitted with either Higuchi equation or Korsmeyer-Peppas equation. Linear or nonlinear fitting was applied on release profiles using software Origin (Orglab8, OriginLab USA). The cumulative release Qₜ at time t is defined as

$${Q}_{t} = \frac{{M}_{t}}{{M}_{∞}} \times 100 \%$$

where \sqrt{Mₜ} is the cumulative amount of drug release at time t and \sqrt{M∞} is the total amount of drug release.
The Higuchi equation is

$${Q}_{t} = A \sqrt{D ( 2 {C}_{0} - {C}_{s} ) {C}_{s} t}$$

where D is the diffusion coefficient, \sqrt{C₀} is the initial drug concentration, \sqrt{Cₛ} is the drug solubility in matrix. The equation can be simplified to

$${Q}_{t} = {K}_{H} {t}^{\frac{1}{2}}$$

The Korsmeyer-Peppas equation is

$${Q}_{t} = {Kt}^{n}$$

where parameter n characterizes the mechanism of release profile. When n<0.45, the drug release mechanism follows Fick's diffusion; when 0.45<n<0.89, it is non-Fick's diffusion, involving skeleton dissolution.

### Procedure for In Vitro Study

Pheochromocytoma 12 (PC12) cell line was used for all the cell tests in this study. The cells were supplied by Dr. T. K. Tang of Institute of Biomedical Sciences, Academia Sinica, Taiwan. Cells were cultured in RPMI-1640 culture media with 10% (v/v) horse serum, 5% (v/v) fetal bovine serum, and 1% (v/v) penicillin/streptomycin/amphotericin B (PSA) at 37° C. under 5% CO₂. The cell seeding density varied for individual experiment and are listed in Table 6. In the case of cell differentiation, the original culture media used to seed cells was removed after 1 day of culturing in RPMI-1640 culture media, and replaced with NGF (100 ng/mL) in RPMI to stimulate the outgrowth of neurites. The culture media with NGF was renewed every 3 days.

TABLE 6
Cell seeding density for different experiments

| Experiment | Cell Density (cells/well) |
|---|---|
| Live/Dead Assay | 20,000 |
| Alamar Blue Assay | 20,000 |
| Cell Differentiation | 5,000 |

### Characterization for Biocompatibility of Scaffolds

The biocompatibility of materials was characterized by using Live/Dead Assay Kit (Molecular Probes). PC12 cells were seeded in sterile 24 wells cell culture plates of polystyrene (cat. #351147, Falcon). At specific time points (1, 5 days), culture media was removed, and samples were washed with PBS solution (Fisher BioReagents) at room temperature. Then cells were stained with 0.05% (v/v in PBS) calcein-AM for live cells and 0.2% (v/v in PBS) ethidium homodimer-1 for dead cells at room temperature for 45 minutes. After staining, solutions were removed, and samples were washed with PBS. The samples were restored in 500 μL PBS and were examined by fluorescence optical microscope (CKX41; OLYMPUS) at 100× magnification. For statistically relevant results, each material was repeated 3 times and 5-6 pictures were taken for cell counting. Cell counting was conducted using ImageJ software (U.S. National Institutes of Health, Bethesda, Maryland, USA).

### Characterization of Cell Viability on Scaffolds

The scaffold effects on cell viability on was analyzed by Alamar Blue Assay IABA) (ABA solution, cat. #BUF102A; AbD Serotec). PC12 cells were seeded at initial density, then at specific time points (1, 3, 5 days), the culture media was removed, and then replaced with 500 μL of 10% Alamar Blue solution (v/v in DMEM) and put into the 37° C., 5% CO₂ incubator for 4 hours. Three wells with ABA solution without cells served as assay blanks. After incubation, the ABA solution was completely removed and then transferred by pipette to a 96-well plate with 100 μL per well. The remaining adherent cells were washed with PBS and fresh culture media was added prior to placement back into incubator. The reacted ABA solution was analyzed with an absorbance microplate reader (800TS; BioTEK). The % reduction of Alamar Blue after the cell culture is calculated according to the following equation:

$$\% reduction of Alamar Blue = \{ [ ( {O}_{1} - {A}_{1} ) - ( {O}_{1} - {A}_{2} ) ] / [ ( {R}_{1} - {N}_{2} ) - ( {R}_{2} - {N}_{1} ) ] \} \times 100 .$$

Here O₁=molar extinction coefficient (E) of oxidized AlamarBlue at 570 nm=80586, O₂=E of oxidized Alamar Blue at 600 nm=117216, R₁=E of reduced Alamar Blue at 570 nm=155677, R₂=E of reduced Alamar Blue at 600 nm=14652, A₁=absorbance of test wells at 570 nm, A₂ =absorbance of test wells at 600 nm, N1=absorbance of negative control well (blank) at 570 nm, N2=absorbance of negative control well (blank) at 600 nm.

### Characterization for Cell Differentiation on Scaffolds

Cells were stained with DAPI and phalloidin for nucleus and F-actin. Formaldehyde (3.7% v/v in PBS) was added to fix cells for 15 minutes at room temperature. After fixation, Triton-X 100 solution (1% v/v in PBS) was added to permeabilize cell membranes for 10 minutes at room temperature. Then, phalloidin solution (1% v/v in PBS) was added to the samples to stain F-actin. After staining for 1 hour at room temperature, DAPI solution (0.2% v/v in PBS) was then added to sample wells to stain cell nuclei for 5 minutes at room temperature. The samples were washed with PBS and restored in PBS for microscopy.

F-actin labeling was used to measure neurite outgrowth. The neurite length was manually measured from the nucleus to the end of the neurite using ImageJ. Each experimental condition was repeated in triplicate, and 77-126 neurites were analyzed.

### Statistical Analysis of Data

In this study, Kruskal-Wallis one way analysis of variance, H-test was used to determine the significant differences between data groups. When p-value <0.05 means significant difference between groups, indicated by *; p-value <0.01 means there is a high degree of difference between groups (highly significant), represent by **; p-value <0.001 means there is an extremely significant difference between groups (extremely significant), represent by *** Differences between groups with p-value >0.05 are not statistically significant and are not marked.

### Synthesis Procedure of Monomer (γ-Benzyl-L-Glutamaic Acid N-Carboxyanhydride, BGNCA)

BGNCA stability testing was as follows 6 g of L-glutamic acid γ-benzyl ester, 90 mL of anhydrous THF, and 7.8 ml of propylene oxide were added and stirred in a three necks round bottom flask for 5 minutes. Then 3.7 g of triphosgene was added, and the solution was stirred for 2 hours at room temperature until clear. The solution was concentrated using a rotary evaporator at 30° C. in order to remove byproducts. Subsequently, 60 ml of anhydrous THF was added, and then n-hexane was added dropwise until precipitation occurred. After overnight crystallization at−20° C., the solution was filtered to collect the precipitation. This recrystallization procedure was repeated three times and the crystals were dried in a vacuum oven at 40° C. A yield of 82% BGNCA was obtained with a molecular weight of 263.25 g/mol.

### Synthesis Procedure of Polymer (Poly(γ-Benzyl-L-Glutamic Acid), PBG))

All three-neck flasks used for polymerization need to be vacuumed overnight to remove moisture before used. Add 3 g of BGNCA into the 100 ml flask and perform three cycles of vacuum and nitrogen purge. Afterward, inject 300 ml of dry benzene to disperse BGNCA. For the polymerization, sodium methoxide was used as the initiator, which was prepared by dissolving 75 mg of sodium in 10 ml of benzene and 5 ml of anhydrous methanol. Initiate polymerization by adding 0.344 ml of sodium methoxide into as-prepared BGNCA solution. After reacting at room temperature for 48 hours in nitrogen atmosphere, the resulting polymer solution was precipitated and filtrated from ethyl acetate, followed by drying in a vacuum oven at 40° C. overnight. A yield of 91% PBG was obtained with Mw=631 kDa, Mn=528 kDa and PDI=1.19.

### Scaffold Fabrication

### Preparation of PBG and PBGMH4 Solution for Electrospinning

The PBG solution for electrospinning was prepared by dissolving PBG in a co-solvent of THF and DMAc (7:3 v/v) overnight to achieve a concentration of 15 wt %. Similarly, the PBGMH4 solution is prepared by incorporating 4 wt % of MH (relate to the amount of PBG) to the same co-solvent of THF and DMAc (7:3 v/v) overnight, and also reaching a solution concentration of 15 wt %.

### Electrospinning of PBG and PBGMH4

The electrospinning setup involved placing a grounded rotating collector at a distance of 10 cm from the needle tip. An applied voltage of 20 kV and a flow rate of 5 ml/hr were employed. The collector was enveloped in aluminum foil and rotated at a speed of 1500 rpm, while fibers were collected on it over a span of around 30 minutes. The final thickness of the resultant felt was within the range of 30 to 50 μm controlled by adjusting the collection time.

### Characterization

The fiber diameter and the cross-section images of PBG and PBGMH4 scaffold was measured by SEM (SU-8010, Hitachi). The porosity of scaffold felt was determined by mercury porosimetry (micromeritics AutoPore IV 9520). The mechanical properties of the scaffold felts were measured by the tensile test machine (YH-H31B7, Yang Yi Technology). The UV—vis absorption spectra (UV-1900, Shimadzu) was used to establish calibration curves (as shown in FIG. 15) of MH in phosphate buffered saline solution (PBS) solution to estimate the cumulative drug release of PBGMH4. The cumulative release of drug over a specific storage month can be calculated by following equation:

$$cumulative release ( \% ) = M\_t / M\_∞ \times 100 \%$$

where Mt stands for the cumulative amount of drug release at time t and Moo denotes the total amount of MH drug.

### Example 4—Use of Scaffold Devices and Systems for Treatments

In some embodiments, the scaffold composition, system and devices may be useful in treating peripheral nerve injury, or PNI. The present example shows that that claimed scaffold can support neurodegeneration by Controlled local delivery of drug. Local drug delivery is superior to systemic administration because lower drug dosages are delivered with greater precision to the affected tissues over a desired period. Effective local drug delivery systems can suppress off-target side effects, attenuate metabolism, or clearance, reduce administration frequency, and improve patient compliance.

### Methods

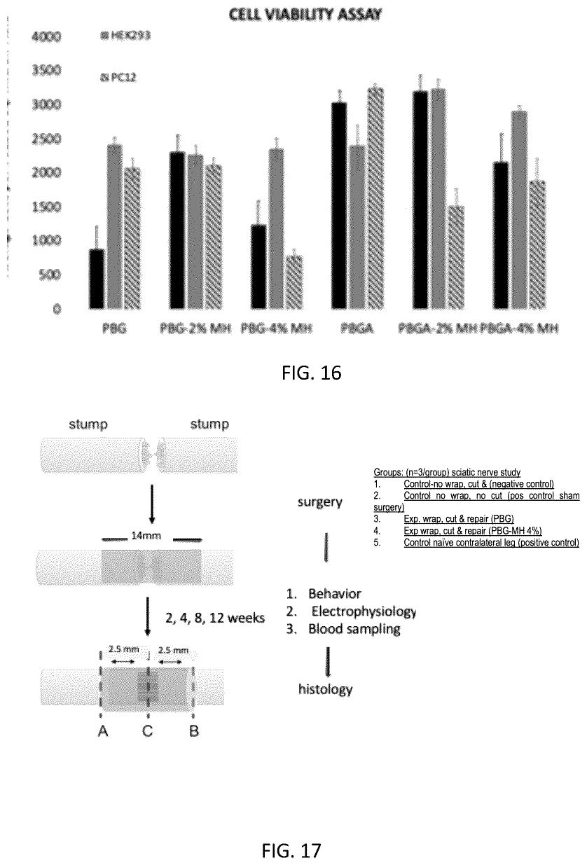

661W & HEK293 note HEK is a epithelial cell line according to literature this demonstrates biocompatibility in other tissues. 293 PC12 & 551W co-cultured with PBG MH scaffolds and assessed by trypan blue for viability after 7 days. (FIG. 16)

2. To test the efficacy of PBG and PBG-MH in vivo we created 4 groups from 12-16 week old male Lewis rats in FIG. 17 (schematic & groups). Surgical procedure & Groups

After preparing the surgical field, the sciatic nerve was exposed according to gluteal-splitting approach and transected with microsurgical scissors. A 14 mm×1.5 mm nerve wrap is sutured using Nylon 8-0 sutures to connect proximal and distal stumps leaving a 9 mm gap between the ends. The incision was closed in layers using 4-0 silk sutures. After surgery, adequate anti-inflammatory (Rimadil, 5 mg/kg) and antibiotic (Bytril, 5 mg/kg) therapies were administered for 5 days and the rats recovered in the cage being housed in a temperature-controlled facility and fed with laboratory rodent diet and water ad libitum. The final end-point was set-up at 12 weeks after surgery; euthanasia by carbon dioxide asphyxiation was performed. Hence, the implants were excised and placed into 4% PFA for downstream IHC analysis. After sciatic nerve transection, rats were allowed to survive for 12 weeks. During this period rats were assayed behaviorally and physiologically. Behavior tests: at PO weeks 2, 4, 6, 8, 10, 12. Tests included: gait analysis and toe spread to generate (SFI), dynamic weight bearing test. CMAP was conducted at PO weeks 4, 8 and 12. Following conclusion of the study, the gastrocnemius muscle was weighed and compared to the contralateral leg for all groups.

3. To test the local delivery of MH to the sciatic nerve, 2 groups of 12-week-old, male Sprague Dawley rats underwent unilateral sciatic nerve transect and repair procedure creating a 10 mm gap. Transacted nerves were repaired with either PCL or PBGA-MH4% wraps (n=2 or 3/group, respectively) as seen in table at right. Prior to surgery, plasma and serum was collected from each animal for baseline comparison. At two weeks post operative (PO), serum and plasma samples were collected from each animal, which was then sacrificed, and the repaired sciatic nerve and proximal samples of caudofemoralis muscle were collected for drug concentration analysis. Implanted scaffolds were found to be highly integrated into the repaired nerve at PO 2 weeks, making their dissection and isolation from the sciatic nerve extremely difficult. For comparison fresh unused scaffolds with equivalent 15×5 mm dimension and drug loading were used to determine concentration of MH delivered to the nerve. Measurement of the correlating decrease in MH concentration from used wraps in vivo was performed as well. Quantification of minocycline by chromatography and mass spectrometry in rats required de novo method development. (FIG. 20)

### Results

1. PBG-MH scaffold was cocultured in 12 well tissue culture dishes with HEK293 cells and assayed after 1 week for toxicity using trypan blue as a vital stain allowing discrimination between viable cells and cells with damaged membranes that are usually considered to be dead cells. HEK293 cells are pre-neuronal origin and we found that cell viability was largely unaffected by coculture with PBG-MH (FIG. 16)

Similarly, mouse 661w retinal cone cells14. and PC12 rat adrenal neurons were largely unaffected with the exception of decreased survival for 661W in PBG and PC12 with PBG-4%. Overall, all cells remained most viable with PBGA and its MH derivatives. (FIG. 16)

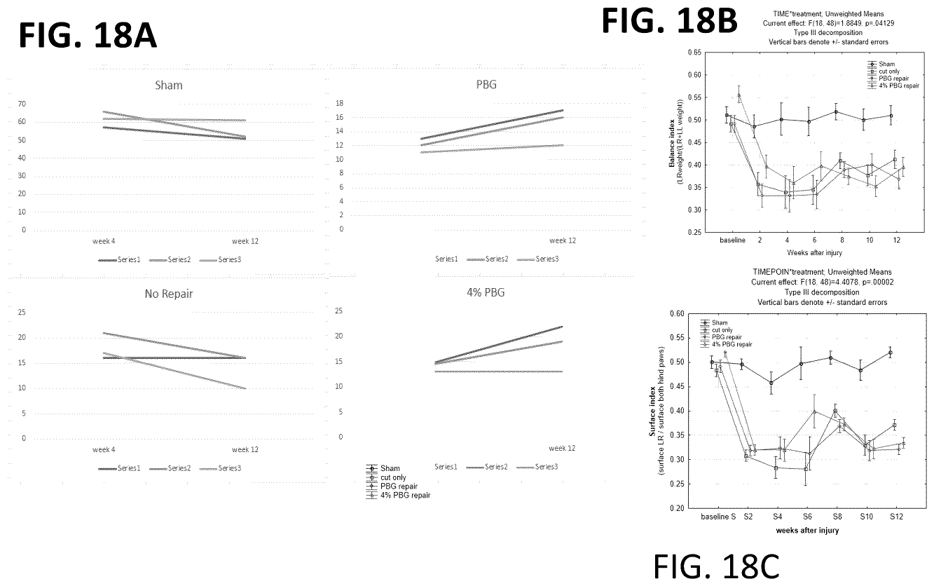

2. we observed an acceleration of neuro-regeneration in the treatment group vs the no treatment group at four weeks post-operatively in both behavioral testing and electrophysiological testing. However, by six weeks the two groups had leveled out in the neuronal function. Additionally, the gastrocnemius muscle appeared to retain more mass at the conclusion of the study, likely the result of earlier and more robust functional recovery. Taken together, the results Summary FIG. 18

2.1 PBG MH 4% improves sciatic nerve functional recovery time a. CMAP response b. dynamic weight bearing test c SFI index calculated by measuring gait and toe spread (FIG. 18)

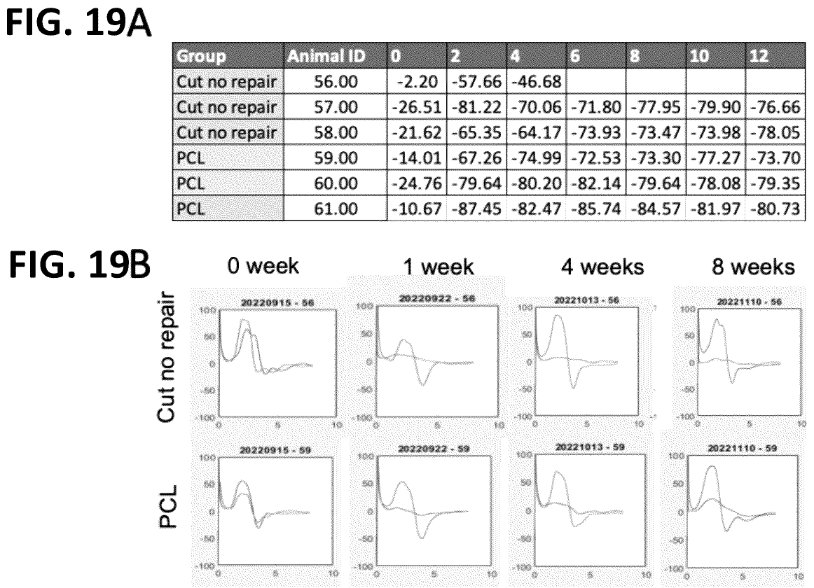

2.2 (FIG. 19A) SFI calculation and CMAP recordings for this group of animals suggest that transection of the sciatic nerve and repair with PCL did not produce significantly better SFI or CMAP as compared to animals from cut without repair group

2.3 FIG. 19B Electrophysiological signals of CMAP examinations carried out 0-12 weeks after implant injured side in each group: TOP PBGMH4 MIDDLE

2.4 PBG MH4 improves GM weight after PNI PBG-MH4% preserves gastrocnemius tissue 12 weeks after sciatic nerve transection (FIG. 20)

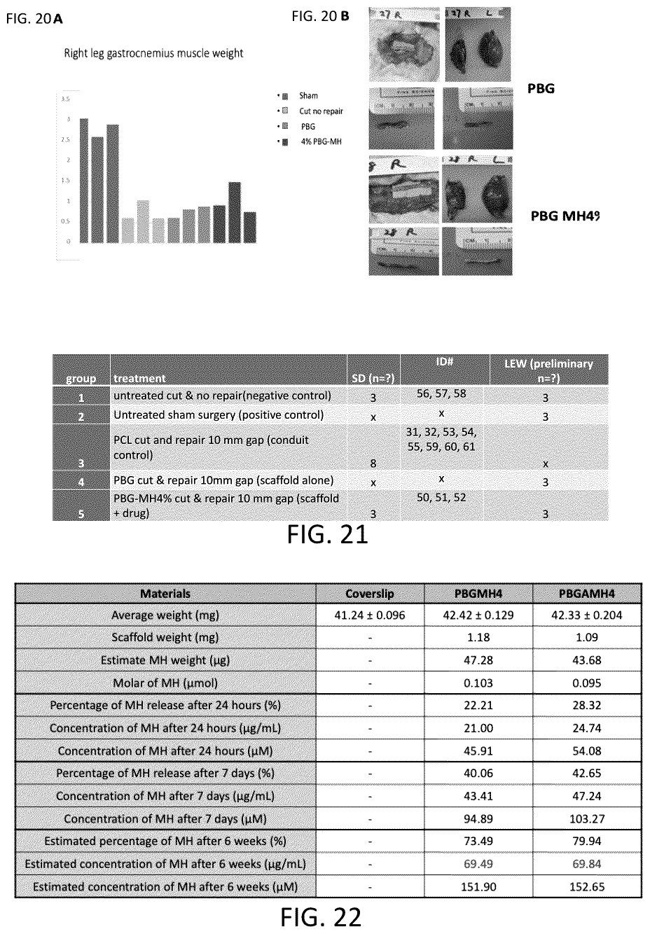

3, Nerve treated with claimed scaffold wrap PBGMH was significantly enriched at Po 2 week vs control wrap, initial concentration or in plasma. (FIG. 21)

A sensitive LC/MS-MS method was prepared for minocycline. The method was linear over a wide concentration range and had low limits of detection and quantitation. The method was used to quantitate drug levels in plasma/serum, nerve tissue, muscle tissue, used wrap and initial drug loaded wrap. The method seems to be rather useful at determining minocycline concentrations from biological samples.

A greater concentration (ng/mL) of MH was detected in the serum vs the plasma of rats, although the drug was detected in both partitions. Collected samples for both fractions have been stored at −80° C., of these samples and for future work, serum will be submitted for assessment of systemic levels of MH introduced by ip or scaffold placement.

At POD 2 weeks, approximately 87% of the MH was release from PBGMH6% scaffolds. Notably, the measured cumulative release of MH from PBG was 42% (FIG. 22), after 7 days in situ. The drug release profiles can be fitted with Higuchi equation and Korsmeyer-Peppas equation demonstrating that the release profile follows Fick's diffusion with little contribution from non-Fick's diffusion, which describes release due to skeleton/scaffold degradation. Theoretical release by PO 6 weeks should be approximately 80% of the total loaded drug. We found about 87% release by PO 2 weeks. This value falls within the fitted linear release model. One major concern, however, is that the initial concentration of MH for individual wraps varied greatly ranging from 10.21-57.01 μg/mg wrap. Such variability calls for increased quality control in electrospinning and manufacturing process to generate robust results that may be attributed to local delivery of MH by the PBG scaffold wrap.

At POD 2-week, sciatic nerve repaired with PBGAMH6% scaffold was ˜68.33 ng/mg. Compared to serum levels of MH at POD 2 week (˜1.6 ng/ml), the represents >40-fold local enrichment of drug to the nervous tissue. Moreover, concentration within the adjacent caudiofemoris tissue was significantly lower at the same timepoint (˜1.47 ng/mg). Altogether, these results support achievement of local drug delivery of MH by PBG scaffold in an in vivo model of sciatic nerve injury.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

We claim:

1. A system for supporting mammalian cells is disclosed, comprising:
a scaffold comprising a plurality of fibers, wherein the majority of fibers are oriented substantially parallel; and
at least one active compound.

2. The system of claim 1, wherein at least one active compound is a growth factor.

3. The system of claim 2, comprising a neuronal cell and the at least one active compound is nerve growth factor.

4. The system of claim 3, wherein the fibers comprise one or more of poly(γ-benzyl-L-glutamate)(PBG), hydrolyzed copolymer (poly(γ-benzylglutamate) 80-r-(γ-glutamic acid)20) (PBGA), and minocycline hydrochloride (MH).

5. A composition comprising:
a polymer comprising at least one trophic factor, and
at least one active compound.

6. The composition of claim 5, the polymer selected from poly(γ-benzyl-L-glutamate) (PBG), and hydrolyzed copolymer (poly(γ-benzylglutamate) 80-r-(γ-glutamic acid)20)(PBGA);
and the at least one active compound is minocycline hydrochloride (MH).

7. The composition of claim 6 fabricated as a fiber.

8. A method of forming a scaffold for growth or support of a mammalian cell, comprising:
combining a polymer selected from poly(γ-benzyl-L-glutamate)(PBG), and hydrolyzed copolymer (poly(γ-benzylglutamate) 80-r-(γ-glutamic acid)20)(PBGA), with at least one active compound, wherein the active compound is minocycline hydrochloride (MH) to form a polymer composition;
forming a fiber from the polymer composition; and
arranging the fiber to create a scaffold.

9. The method of claim 8, wherein the forming and arranging are accomplished using electro spinning.

10. The method of claim 9, wherein the concentration of the active compound is less than about 5% by weight.

11. A method of treating a peripheral nerve injury, comprising:
identifying a mammalian subject having an injury to a cell or tissue;
surrounding the injured cell or tissue with the composition of claim 7.

12. The method of claim 11, wherein the injury is to a sciatic nerve.

13. A device comprising:
fiber scaffold comprising;
a polymer; and
an active compound.

14. The device of claim 13, wherein the polymer is selected from poly(γ-benzyl-L-glutamate) (PBG), and poly(γ-benzylglutamate) 80-r-(γ-glutamic acid) 20 (PBGA).

15. The device of claim 14, wherein the polymer PBGA.

16. The device of claim 15, wherein the active compound is minocycline hydrochloride (MH).

17. The device of claim 16, wherein the active compound is less than about 5% by weight of the fiber.

18. The device of claim 17, wherein release of the active compound from the fiber does not show two-phase kinetics after 24 hours.

19. The device claim 18, wherein at least 50% of the fibers are oriented in the same or the opposite direction.

20. The device of claim 19, for use in treating a peripheral or central nervous system cell or tissue.
